(19) United States
(12) Patent Application Publication
(10) Pub. No.: US 2010/0248265 A1
(43) Pub. Date: Sep. 30, 2010

(54) COMPOSITIONS AND METHODS FOR DIAGNOSIS AND TREATMENT OF CANCER

(75) Inventors: Tony Hunter, Del Mar, CA (US); Jeremy T. Copp, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Salk Institute for Biological Studies, La Jolla, CA (US)

(21) Appl. No.: 12/715,322

(22) Filed: Mar. 1, 2010

Related U.S. Application Data
(60) Provisional application No. 61/156,293, filed on Feb. 27, 2009.

(51) Int. Cl. G01N 33/53

(52) U.S. Cl. 435/7.8

(57) ABSTRACT

Provided herein are methods and compositions useful in the diagnosis and treatment of cancer. The methods and compositions typically involve detecting a level of phosphorylation of mTOR at serine 2481 in a subject and comparing the level of phosphorylation of mTOR at serine 2481 in said subject with a standard control.

### CROSS-REFERENCES TO RELATED APPLICATIONS

This application claims the benefit of U.S. Provisional Application No. 61/156,293, filed Feb. 27, 2009, the contents of which are incorporated herein by reference in its entirety for all purposes.

### STATEMENT AS TO RIGHTS TO INVENTIONS MADE UNDER FEDERALLY SPONSORED RESEARCH OR DEVELOPMENT

This invention was made with government support under CA82683 and CA14195 awarded by the National Cancer Institute and T32 CA 09370 awarded by the National Institutes of Health. The Government has certain rights in the invention.

### BACKGROUND OF THE INVENTION

Many diseases are characterized by disruptions in cellular signaling pathways that lead to pathologies including uncontrolled growth and proliferation of cancerous cells, as well as aberrant inflammation processes. Such defects include changes in the activity of lipid kinases, a class of enzymes that catalyze the transfer of phosphate groups to lipids. These phosphorylated lipids, in turn, recruit important downstream proteins that propagate the signals originating from upstream signaling mediators, such as receptor tyrosine kinase and antigen receptors. For example, the protein kinase Akt is recruited by phospholipids to the plasma membrane where it is activated. Both Akt and the Ser/Thr kinase mTOR play pivotal roles in the survival both of normal and cancerous tissues.

mTOR is a member of the PI-3 kinase-like kinase family (PIKKs) that plays an integral role in coordinating cell growth and division in response to growth factors, nutrients and the energy status of the cell. mTOR is found in two distinct signaling complexes that are evolutionarily conserved from yeast to mammals. These complexes have differing substrate specificity that is determined by the unique mTOR-interacting proteins that are found in each complex. The rapamycin-sensitive mTORC1 contains mTOR, Raptor, mLST8 and PRAS40 (Hara et al., Cell, 110:177-89 (2002); Kim et al., Cell, 110:163-75 (2002); Kim et al., Mol Cell, 11:895-904 (2003); Haar et al., Nat Cell Biol, 9:316-23 (2007)), and regulates cell growth and translation in part by phosphorylating S6K and the eIF-4E binding protein 1 (4E-BP1) (Tee, A. R., Blenis, J., Semin Cell Dev Biol, 16:29-37 (2005)). The rapamycin-insensitive mTORC2 contains mTOR, Rictor, mSin1, mLST8 and Protor (Sarbassov et al., Curr Biol, 14:1296-302 (2004); Frias et al., Curr Biol, 16:1865-70 (2006); Jacinto et al., Cell, 127:125-37 (2006); Yang et al., Genes Dev, 20:2820-32 (2006); Pearce et al., Biochem J, 405:513-22 (2007)). In select tumor cell lines, mTORC2 is sensitive to prolonged rapamycin treatment, which inhibits mTORC2 assembly and function (Sarbassov et al., Mol Cell, 22:159-68 (2006)). mTORC2 regulates organization of the actin cytoskeleton through the phosphorylation of PKCα, and also phosphorylates and activates Akt at the hydrophobic motif (HM) site, S473 (Sarbassov et al., Curr Biol, 14:1296-302 (2004); Sarbassov et al., Science, 307:1098-101 (2005)). Although several other kinases have been reported to phosphorylate Akt at S473, including the PIKK-family members DNA-PK and ATM (Feng et al., J Biol Chem, 279:41189-96 (2004); Viniegra et al., J Biol Chem, 280:4029-36 (2005); Bozulic et al., Mol Cell, 30:203-13 (2008)), genetic evidence from Rictor, mSin1 and mLST8 knockout mice demonstrates that intact mTORC2 is necessary for maximal phosphorylation and activation of Akt in mouse embryos, suggesting it is the major S473 kinase under normal conditions (Jacinto et al., Cell, 127:125-37 (2006); Shiota et al., Dev Cell, 11:583-9 (2006); Guertin et al., Dev Cell, 11:859-71 (2006)). Nevertheless, DNA-PK may be an important regulator of S473 phosphorylation in response to genotoxic stress and DNA damage (Bozulic et al., Mol Cell, 30:203-13 (2008)).

Upon activation, mTOR is phosphorylated on several residues, including T2446, S2448 and S2481. T2446 is phosphorylated in response to nutrient availability (Cheng et al, J Biol Chem, 279:15719-22 (2004)). Initially, S2448 was reported to be an Akt phosphorylation site because its phosphorylation is sensitive to PI-3 kinase (PI-3K) inhibition, which reduces Akt activity. However, more recent reports have shown that S6K is the S2448 kinase (Chiang et al., J Biol Chem, 280:25485-90 (2005); Holz et al., J Biol Chem, 280:26089-93 (2005)). S2481 is a rapamycin-insensitive autophosphorylation site (Peterson et al., J Biol Chem, 275:7416-23 (2000)). All three phosphorylation sites are in a region lying between the catalytic domain and the FATC domain near the C-terminus of mTOR. Mutation of T2446 and S2448 to alanine has no discernible effect on the ability of mTOR to activate its downstream effectors. Internal deletion of residues 2430-2450 reportedly increases mTOR kinase activity (Sekulic et al., Cancer Res, 60:3504-13 (2000)).

The nutrient responsive signaling pathways, including the mTOR pathway, are critical in oncogenesis, particularly solid tumor and hematological malignancies. mTOR is a serine/threonine kinase responsible for cell proliferation/survival signaling by inducing cell-cycle progression from G1 to S phase in response to nutrient availability, (Maloney and Rees, Reproduction, 130:401-410 (2005)). Dysregulation in the mTOR signaling pathway has been linked to oncogenesis. The mTOR pathway includes multiple small molecule targets for therapeutic intervention. mTOR inhibitors have been developed including rapamycin and its analogues CCI-779, RADOO1, and AP23573. Such treatments are currently in phase II-III clinical trials (Janus, et al., Cell MoI Biol Lett, 10(3):479-98 (2005)).

Although a limited number of mTOR phosphorylation sites are known, and a few antibodies for their study available, there remains a need for the identification of additional phosphorylation sites relevant to activity of this kinase. Accordingly, new and improved reagents and methods for the detection of mTOR activity would be desirable, including development of reagents against newly identified sites of mTOR phosphorylation. Since phosphorylation-dependent over-activation of mTOR is associated with diseases such as lymphoma, glioma, and colon cancer, reagents enabling the specific detection of mTOR activation would be useful tools for research and clinical applications. Solutions to these and other problems in the art are provided herein.

### BRIEF SUMMARY OF THE INVENTION

In one aspect, a method of predicting whether a subject that has a cancer would be responsive to an mTor inhibition cancer treatment is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates the subject would be responsive to an mTOR inhibition cancer treatment.

In another aspect, a method of monitoring progression of a cancer in a subject that has the cancer is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates a higher progression of cancer in the subject.

In another aspect, a method of determining whether a subject is at risk of developing a cancer is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates the subject is at risk of developing the cancer.

In another aspect, a method of determining whether a subject has a cancer is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates the subject has the cancer.

In some embodiments of one or more of the aspects described above, the detecting the level of phosphorylation of mTOR at serine 2481 in the subject includes detecting a level of phosphorylation of mTOR at serine 2481 in a sample from the subject. The detecting the level of phosphorylation of mTOR at serine 2481 in the subject may include contacting the sample with an anti-S2481 antibody.

In another aspect, a method is provided of determining whether a test compound is a cancer therapeutic. The method includes contacting the test compound with a cell. A level of phosphorylation of mTOR at serine 2481 in the cell is detected. The level of phosphorylation of mTOR at serine 2481 is compared to a standard control. A high level of phosphorylation of mTOR at serine 2481 in the cell relative to the standard control indicates the test compound is a cancer therapeutic.

### BRIEF DESCRIPTION OF THE DRAWINGS

FIG. 1 provides a schematic and images showing mTORC1 and mTORC2 contain differentially phosphorylated mTOR. (A) Schematic of mTOR and multiple sequence alignment of the C-terminus of selected vertebrate and invertebrate mTORs. Invertebrate species are Ciona intestinalis and C. savignyi (Cint and Csav); Drosophila melanogaster and D. virilis (Dmel and Dvir); Caenorhabditis elegans and C. briggsae (Cele and Cbrig); and Saccharomyces cerevisiae (mTOR1 and mTOR2). The region between the kinase domain (KD) and an N-terminal extension of the FATC domain (N-FATC) is conserved among vertebrates, including the marked phosphorylation sites S2448 and S2481. Asterisks indicate residues completely conserved in vertebrates. (B) HEK293 cells were serum-starved overnight. The indicated cells were stimulated with 200 nM insulin for 5 min at 37° C. Rictor and Raptor immunoprecipitates (IPs) from control and growth factor-stimulated cells were analyzed by immunoblotting with antibodies specific for mTOR phosphorylated on S2448 or S2481, or total mTOR. Whole cell lysates (WCL) were included as controls for total input. (C) Samples from actively growing U2OS cells were analyzed as in (B). Results are representative of multiple independent experiments.

FIG. 2 shows intact mTORC1 and mTORC2 are necessary for mTOR phosphorylation. (A) HEK293 cells were infected with lentiviruses expressing a control shRNA or shRNAs targeting mTOR, Raptor or Rictor. Cells were selected with puromycin 24 hr. after infection and then serum-starved overnight 2 days post-selection. The indicated cells were stimulated with 200 nM insulin for 5 min. at 37° C. WCLs were normalized for total protein concentration and were analyzed by immunoblotting with antibodies specific for mTOR phosphorylated on S2448 or S2481. Blots for total mTOR, Rictor and Raptor were included as controls. (B) HEK293 cells were infected with lentiviruses expressing a control shRNA or shRNAs targeting Rictor or mSin1. Cells were treated as in (A) and analyzed by immunoblotting for mTOR phosphorylated on S2448 or S2481. Blots for total mTOR, Rictor and mSin1 were included as controls. (C) Wild type (WT) and Sin1^{-/-} mouse embryo fibroblasts (MEFs) were serum starved overnight. The indicated cells were stimulated with 200 nM insulin for 5 min. at 37° C. WCLs were normalized for total protein concentration and were analyzed by immunoblotting with antibodies specific for mTOR phosphorylated on S2481, total mTOR and Rictor. (D) HEK293 cells were infected with lentiviruses and were treated as in (A). Rictor and Raptor IPs from control and growth factor-stimulated cells were analyzed by immunoblotting for bound mTOR. WCLs were analyzed by immunoblotting with antibodies specific for mTOR phosphorylated on S2448 or S2481. Results are representative of multiple independent experiments.

FIG. 3 shows prolonged treatment of cells with rapamycin inhibits mTOR phosphorylation on S2448 and S2481. (A) Serum-starved HEK293 cells were cultured in the presence or absence of 100 nM rapamycin for either 1 or 24 hr. The indicated cells were stimulated with 200 nM insulin for 5 min. at 37° C. WCLs were analyzed by immunoblotting with antibodies specific for mTOR phosphorylated on S2448 or S2481, or total mTOR. Rictor IPs were analyzed by immunoblotting with antibodies specific for Rictor, mTOR, and mTOR phosphorylated on S2481. (B) Actively growing U2OS cells were cultured in the presence or absence of 100 nM rapamycin for either 1 or 24 hr and analyzed as described as in (A). (C) Actively growing MDA 231, MDA 468, SKBR3 and A549 cells were treated as in (B). Rictor IPs were analyzed by immunoblotting with antibodies specific for Rictor and mTOR. WCLs were analyzed by immunoblotting for phospho-mTOR(S2481), phospho-Akt (S473), phospho-S6K (T389), total mTOR and total Akt. (D) Actively growing C2C12 myoblasts and HepG2 cells were treated as in (B) and WCLs were analyzed as in (C). Results are representative of multiple independent experiments.

FIG. 4 shows depletion of mTORC2 renders S473 phosphorylation of Akt sensitive to rapamycin. MDA-MB-468 cells were infected with lentiviruses expressing a control shRNA or shRNAs targeting mTOR, Rictor or mSin1. Cells were selected with puromycin 24 hr. after infection and the indicated cells were treated with 100 nM rapamycin for an additional 24 hr. WCLs were normalized for total protein concentration and were analyzed by immunoblotting for phospho-mTOR(S2481), phospho-Akt (S473), phospho-S6K (T389), and total Akt, mTOR Rictor and mSin1. Results are representative of multiple independent experiments.

FIG. 5 shows that S2481 phosphorylation is a marker of mTOR activity in response to mTOR kinase inhibitors. Whole cell lysates from control cell or cells treated with either Torinl, PP242 or PIK-90 were analyzed by western blotting with antibodies that recognize mTOR phosphorylated on S2481, S6K phosphorylated on T389 or Akt phosphorylated on S473.

FIGS. 6A-I show phospho-S2481 staining in section from human patients with invasive ductal carcinoma. (A) Staining of tumor tissue from a stage I invasive ductal carcinoma shows higher levels of phospho-S2481 in the indicated DCIS (arrow). (B) Higher magnification of the indicated area from panel A. (C) Higher magnification of the DCIS. These cells exhibit larger nuclei with hyperchromasia in comparison to the surrounding cells. (D) Staining of tumor tissue from a stage IIb invasive ductal carcinoma showing higher levels of phospho-S2481 in multiple ducts with DCIS with mixed growth patterns. (E) Higher magnification tissue from panel D. Some cells are no longer in contact with the surrounding stroma (arrow), which indicates invasiveness. (F) Higher magnification showing a cribiform growth pattern of hyperchromic tumor cells in this duct. (G) Staining of tumor tissue from a stage IIIa invasive ductal carcinoma demonstrating higher levels of phospho-S2481 in the invasive tumor cells. (H) Higher magnification of the tissue in panel G. The tumor tissue displays minimal tubule formation. (I) Higher magnification showing abnormal, poorly differentiated breast cells that appear to be more aggressive.

FIGS. 7A-I show phospho-S473 staining in sections from human patients with invasive ductal carcinoma. The tumor tissue section described in FIGS. 6A-I were stained for phospho-Akt, a substrate of mTORC2. These data confirm that areas with high mTOR activity, as deduced by phospho-S2481 staining, have high levels of Akt that is phosphorylated on S473.

FIGS. 8A-I show phospho-T389 staining in section from human patients with invasive ductal carcinoma. The tumor tissue section described in FIGS. 6A-I were stained for phospho-S6K, a substrate of mTORC1. These data confirm that areas with high mTOR activity, as deduced by phospho-S2481 staining, have high levels of S6K that is phosphorylated on T389.

FIGS. 9A-C show phospho-S2481 staining of invasive ductal carcinoma cases contained in a breast tumor tissue array. (A) Normal breasts tissue. (B) Stage IIIb invasive ductal carcinoma. (C) Stage I invasive ductal carcinoma.

FIG. 10 shows that S2481 phosphorylation is a marker of mTOR activity in response to mTOR kinase inhibitors. Whole cell lysates from control cell or cells treated with either Torin1 or PP242 were analyzed by western blotting with antibodies that recognize mTOR phosphorylated on S2481, S6K phosphorylated on T389 or Akt phosphorylated on S473.

FIGS. 11A-F show phospho-S2481 staining in section from human patients with invasive lung adenocarcinoma. (A) Staining of a moderately differentiated adenocarcinoma of the lung. (B) Higher magnification of the indicated area from panel A. The glands are relatively well formed and lined with atypical epithelial cells that infiltrate the surrounding stroma. (C) Higher magnification of the indicated area from panel B. These cells are tall and columnar, and many have basally situated, larger nuclei with hyperchromasia. The cytoplasm of these cells contains higher levels of phospho-S2481 compared to the surrounding stroma. See panel B. (D) Staining of a moderately to poorly differentiated adenocarcinoma of the lung. (E) Higher magnification of the indicated area from panel D. The glandular structures are poorly formed in comparison to panel B. (F) Higher magnification of the indicated area from panel E. These cells exhibit larger nuclei with more hyperchromasia and some loss of nuclear polarity in comparison to panel C.

FIGS. 12A-C show phospho-S2481 staining in sections from the K-ras X LKB1+− mouse model of lung cancer. (A) Staining of a lung displaying hyperplasia. (B) Higher magnification of the indicated area from panel A. This area is typical of an adenoma, consisting of a uniform population of epithelial cells with relatively round nuclei. Both the nuclei and the cytoplasm of these cells are stained for S2481 phosphorylation as indicated by the brown color in the original micrograph which is shown in grayscale in the figure. (C) Higher magnification of the indicated area from panel A. These cells more closely resemble those found in an adenocarcinoma, showing greater cytological atypia with more variation in regional growth patterns. These cells do not show the high degree of staining for phospho-S2481 seen in panel B.

FIGS. 13A-C show that phospho-S2481 are significantly decreased in section from the K-ras X LKB1−/− mouse model of lung cancer. (A) Staining of this section shows a severe decrease in S2481 phosphorylation when compared to FIGS. 11A-F. (B) Higher magnification of the indicated area from panel A. This area consists of cells indicative of an adenocarcinoma. They demonstrate cytological atypia and more variation in regional growth. These is minimal staining for phospho-S2481. (C) Higher magnification of the indicated area from panel A. This hyperplasia is also an adenocarcinoma, with levels of phospho-S2481 comparable to those seen in FIG. 12C.

FIG. 14 shows that mTOR is phosphorylated on S2481 in A549 cells reconstituted with LKB 1. Whole cell lysates from A549 cells reconstituted with an empty control expression construct or an expression construct for LKB1 were analyzed by western blotting with antibodies that recognize mTOR phosphorylated on S2481 and total mTOR.

FIG. 15, A and B show the sequence of a human mTOR protein.

### DETAILED DESCRIPTION OF THE INVENTION

### I. Definitions

As used herein, the term “cancer” refers to all types of cancer, neoplasm or malignant tumors found in mammals, including leukemia, carcinomas and sarcomas. Exemplary cancers include cancer of the brain, breast, cervix, colon, head & neck, liver, kidney, lung, non-small cell lung, melanoma, mesothelioma, ovary, sarcoma, stomach, uterus and Medulloblastoma. Additional examples include, Hodgkin's Disease, Non-Hodgkin's Lymphoma, multiple myeloma, neuroblastoma, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine and exocrine pancreas, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number abnormal cells in the blood-leukemic or aleukemic (subleukemic). The P₃₈₈ leukemia model is widely accepted as being predictive of in vivo anti-leukemic activity. It is believed that a compound that tests positive in the P₃₈₈ assay will generally exhibit some level of anti-leukemic activity in vivo regardless of the type of leukemia being treated. Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophylic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, multiple myeloma, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Sarcomas include chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniformi carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

### II. Methods

Provided herein, inter alia, are methods and reagents for determining or predicting response to cancer therapy in an individual; methods for using image analysis of immunohistochemically-stained samples to quantify gene expression, phosphorylation, or both for genes of cancer-related metabolic pathways, including mTOR phosphorylation (activation); therapeutic treatments directed to such cancer-related metabolic pathways; methods for predicting response in cancer subjects to cancer therapy, including cancer patients; predictive biomarkers to identify those cancer patients for whom administering a therapeutic agent will be most effective; predictive biomarkers for assessing or monitoring the efficacy of therapeutic agents targeted to mTOR pathway; kits for identifying a mammalian tumor in need of or assessing a response in a subject to receiving a mTOR pathway inhibitor; and mTOR inhibitor therapeutic treatment.

In one aspect, a method of predicting whether a subject that has a cancer would be responsive to an mTOR inhibition cancer treatment is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates the subject would be responsive to an mTOR inhibition cancer treatment. In some embodiments, the method of predicting whether a subject that has a cancer would be responsive to an mTOR inhibition cancer treatment is a method of determining whether a subject that has a cancer is likely to be responsive to an mTOR inhibition cancer treatment. The term “responsive to an mTOR inhibition cancer treatment,” as used herein, means slowing or halting the pathogenic processes (e.g. growth of cancer cells) that lead to the cancer progression and/or the cancer symptomatic effects in the subject and/or an improvement in the symptoms of the subject (e.g. killing most or all of the cancer cells within the subject). In some embodiments, where a high level of phosphorylation of mTOR at serine 2481 is detected in the subject, the method further includes treating said subject with said mTOR inhibition cancer treatment (e.g. administering to the subject an effective amount (e.g. a therapeutically effective amount) of an mTOR inhibitor).

In another aspect, a method of monitoring progression of a cancer in a subject that has the cancer is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates a higher progression of cancer in the subject. In some embodiments, the method of monitoring progression of the cancer is a method of assessing the stage or severity of the cancer (e.g. a determination of clinical severity of the cancer). In some embodiments, where a high level of phosphorylation of mTOR at serine 2481 is detected in the subject, the method further includes treating said subject with an mTOR inhibition cancer treatment (e.g. administering to the subject an effective amount (e.g. a therapeutically effective amount) of an mTOR inhibitor).

In another aspect, a method of determining whether a subject is at risk of developing a cancer is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates the subject is at risk of developing the cancer. The risk may be assessed using any appropriate reporting technique, including an assessment of the likelihood of developing the cancer in terms of overall percentage or risk over time. In some embodiments, where a high level of phosphorylation of mTOR at serine 2481 is detected in the subject, the method further includes treating said subject with an mTOR inhibition cancer treatment (e.g. administering to the subject an effective amount (e.g. a therapeutically effective amount) of an mTOR inhibitor).

In another aspect, a method of determining whether a subject has a cancer is provided. The method includes detecting a level of phosphorylation of mTOR at serine 2481 in the subject. The level of phosphorylation of mTOR at serine 2481 in the subject is compared with a standard control. A high level of phosphorylation of mTOR at serine 2481 in the subject relative to the standard control indicates the subject has the cancer. A subject that “has a cancer” is a cancer subject (e.g. a cancer patient). A cancer subject may or may not display clinical symptoms of the cancer, but the cancer subject does contain cancer cells. In some embodiments, where a high level of phosphorylation of mTOR at serine 2481 is detected in the subject, the method further includes treating said subject with an mTOR inhibition cancer treatment (e.g. administering to the subject an effective amount (e.g. a therapeutically effective amount) of an mTOR inhibitor).

In some embodiments of one or more of the aspects described above, the detecting the level of phosphorylation of mTOR at serine 2481 in the subject includes detecting a level of phosphorylation of mTOR at serine 2481 in a sample from the subject. The detecting the level of phosphorylation of mTOR at serine 2481 in the subject may include contacting the sample with an anti-S2481 antibody.

In some embodiments of one or more of the aspects described above, the cancer is breast cancer or lung cancer. The subject may be a mammalian subject, such as a human subject. In some embodiments, the human subject is a patient.

“A high level of phosphorylation of mTOR at serine 2481 in the subject relative to the standard control” as used herein, means that the determined level of phosphorylation of mTOR at serine 2481 is elevated relative to the standard control (also referred to herein as a “control”). A person having ordinary skill in the art will recognize that even where the absolute value of the determined level of phosphorylation of mTOR at serine 2481 is lower than the absolute value of the control the level of phosphorylation of mTOR at serine 2481 may still be considered considered high. For example, in some embodiments, the level of phosphorylation of mTOR at serine 2481 may be the same or less than the control and be considered high depending upon the type of control employed. It is within the capabilities of a person having ordinary skill in the art using the teachings provided herein to determine the type of control employed and to determine whether the resulting level of phosphorylation of mTOR at serine 2481 is considered high relative to the control employed.

For example, in some embodiments of one or more of the aspects described above, the standard control is a level of phosphorylation of mTOR at serine 2481 in the subject determined at an earlier time point (e.g. when the subject was known to be healthy). Where the level of phosphorylation of mTOR at serine 2481 in the subject is sufficiently higher than the standard control level of phosphorylation of mTOR at serine 2481 in the subject at an earlier time point when the subject was considered healthy (e.g. cancer free), the level of phosphorylation of mTOR at serine 2481 in the subject relative to the standard control is considered high. Where the level of phosphorylation of mTOR at serine 2481 in the subject is sufficiently lower than the standard control level of phosphorylation of mTOR at serine 2481 in the subject at an earlier time point when the subject was considered unhealthy (e.g. the subject had cancer), the level of phosphorylation of mTOR at serine 2481 in the subject relative to the standard control is considered low.

In other embodiments of one or more of the aspects described above, the standard control is an average level of phosphorylation of mTOR at serine 2481 derived (e.g. obtained from and possibly subsequently processed for testing) from a plurality of control subjects. The controls subjects may be cancer subjects. Where the control subjects are cancer subjects, the level of phosphorylation of mTOR at serine 2481 in the subject may be considered high where the level is approximately equal to the average level of phosphorylation of mTOR at serine 2481 in samples derived from the plurality of cancer subjects. In other embodiments, the control subjects may be healthy subjects. Where the control subjects are healthy subjects, the level of phosphorylation of mTOR at serine 2481 in the subject may be considered high where the level is sufficiently higher than the average level of phosphorylation of mTOR at serine 2481 in samples derived from the plurality of healthy subjects.

“An mTOR inhibition cancer treatment,” as used herein, refers to a treatment for a cancer in which mTOR activity is decreased relative to an amount of mTOR activity in the absence of the treatment. In some embodiments of one or more of the aspects described above, the mTOR inhibition cancer treatment is a treatment in which a therapeutically effective amount of an mTOR inhibitor is administered to the subject. The mTOR inhibitor may be, for example, rapamycin, Ku-0063794, PP242, PP30, Torin1 or analogs thereof. Rapamycin and its analogs (e.g. CCI-779, RADOO1, and AP23573), Ku-0063794 (re1-5-[2-[(2R,6S)-2,6-Dimethyl-4-morpholinyl]-4-(4-morpholinyl)pyrido[2,3-d]pyrimidin-7-yl]-2-methoxybenzenemethanol), PP242, PP30, and Torin1 are well known in the art (see, e.g., Thoreen C C, et al. J Biol Chem, 2009 Jan. 15; Feldman M E, et al. PLoS Biol. 2009 Feb. 10; 7(2):e38; and Garcia-Martinez, J., et al. 2009, Biochem J 421: 29-42).

In another aspect, a method is provided of determining whether a test compound is a cancer therapeutic. The method includes contacting the test compound with a cell. A level of phosphorylation of mTOR at serine 2481 in the cell is detected. The level of phosphorylation of mTOR at serine 2481 is compared to a standard control. A high level of phosphorylation of mTOR at serine 2481 in the cell relative to the standard control indicates the test compound is a cancer therapeutic. In some embodiments, the cell is a mammalian cell (e.g. a human cell).

In some embodiments, the standard control is a level of phosphorylation of mTOR at serine 2481 in the cell in the absence of the test compound.

The methods disclosed herein may be employed with any appropriate sample (e.g. a biological sample). In some embodiments, the sample is suspected of containing phosphorylated mTOR, more specifically mTOR phosphorylated at serine 2481. Biological samples taken from human subjects may be any appropriate sample, including tissue, fluids, biopsy samples and the like. In some embodiments, the tissue is a cancer tissue, such as breast cancer tissue, lung cancer tissue, lymphoma, glioma, and colon cancer tissue, suspected of involving altered mTOR phosphorylation. In other embodiments, the sample is urine, stool, serum, blood plasma, fine needle aspirate, ductal lavage, bone marrow sample or ascites fluid. Although the present application is primarily concerned with the treatment of human subjects, the disclosed methods may also be used with other mammalian subjects such as dogs and cats for veterinary purposes.

“A level of phosphorylation of mTOR at serine 2481 in a subject,” as used herein, refers to an amount (e.g. a number, a percentage of total protein, a percentage of total mTOR protein, or some appropriate measure of a number or percentage) of mTOR in a subject (e.g. in a sample derived from a subject) in which the mTOR is phosphorylated at serine 2481. The mTOR phosphorylated at serine 2481 typically forms part of the mTORC2 complex rather than the mTORC1 complex. Therefore, in some embodiments, the level of phosphorylation of mTOR at serine 2481 in a subject is the level of phosphorylation of mTOR at serine 2481 in a subject wherein the mTOR forms part of the mTORC2 complex (i.e. mTOR is associated with other protein subunits of the mTORC2 complex). In some embodiments, the mTOR phosphorylated at serine 2481 is detected only when complexed with Rictor. In other embodiments, the mTOR phosphorylated at serine 2481 is detected only when complexed with Rictor, mSin1, mLST8 and/or Protor. In other embodiments, the mTOR phosphorylated at serine 2481 is detected only when complexed with Rictor, mSin1, mLST8 and Protor. In some embodiments, the phosphorylation of mTOR at serine 2481 is autophosphorylation of mTOR at serine 2481.

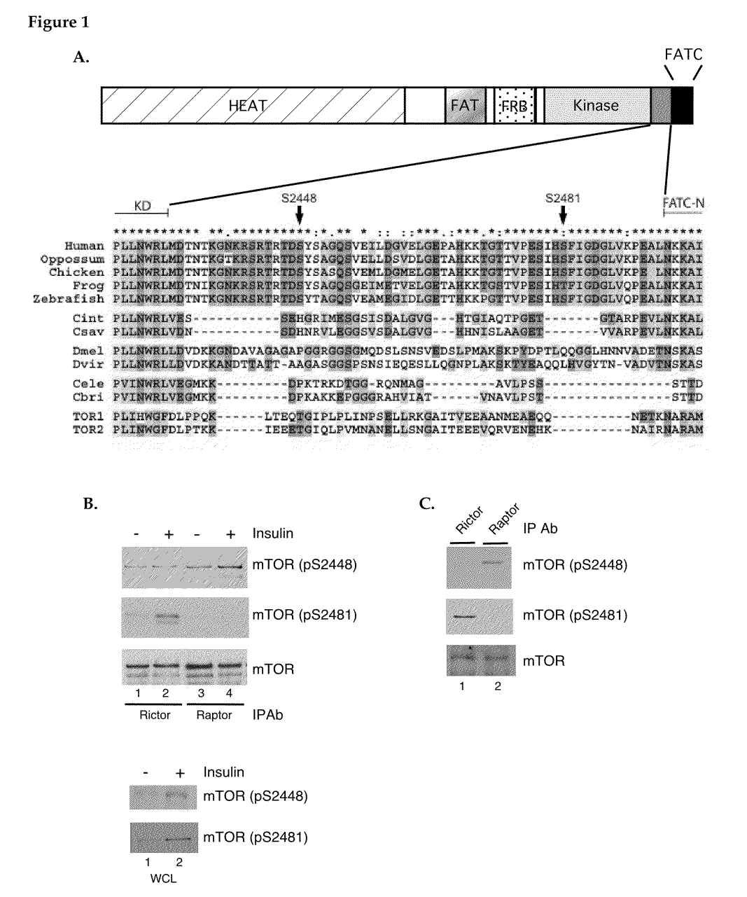

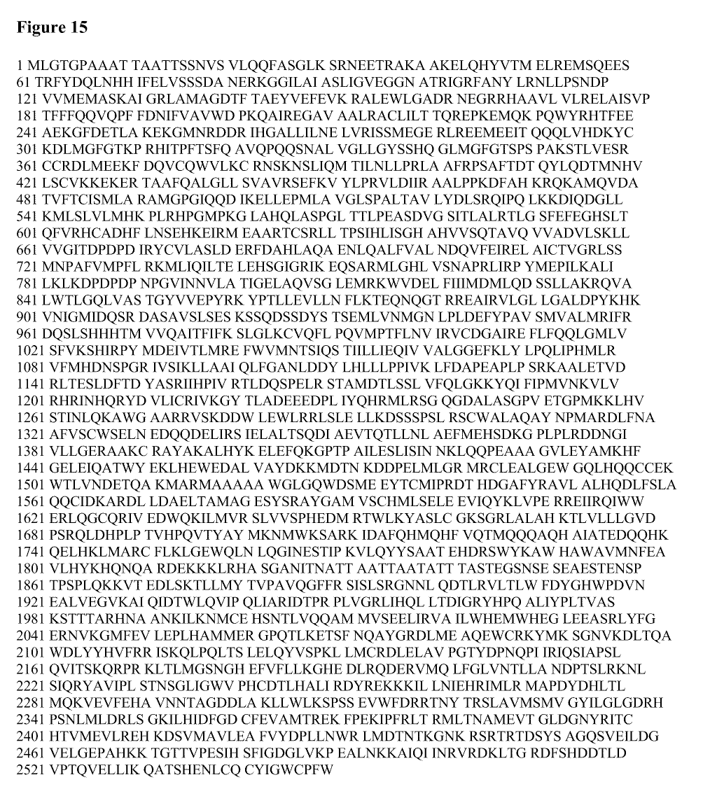

“Serine 2481,” as used herein, refers to a serine at position 2481 of the human mTOR protein sequence (Accession No. P42345), or fraction thereof, as set forth for example in FIGS. 1 and 15 (see FIG. 1.A. Human sequence), or equivalent serine in an mTOR homolog of the human sequence (see e.g. FIG. 1.A listing certain mTOR homolog sequences with equivalent serines). The phrase “in a subject” within the term “a level of phosphorylation of mTOR at serine 2481 in a subject,” as used herein, refers to a subject or an appropriate proxy for the subject such as a sample derived (e.g. obtained) from the subject (e.g. a biological sample such as a liquid sample or tissue sample as discussed herein). The term “mTOR,” refers to an mTOR protein (see Accession No. P42345, see also FIG. 15), fractions or subunits thereof (e.g. FIG. 1.A. Human sequence), and homologs thereof (e.g. FIG. 1.A) having a serine 2481 (e.g. proteins including the mTOR sequences with S2481 in FIG. 1.A). Where the subject is a human subject, the mTOR is a human mTOR protein or fraction or subunit thereof. The mTOR protein may have the amino acid sequence set forth in FIG. 15, and includes homologs thereof, fractions thereof, or subunits thereof.

Detecting phosphorylated mTOR at serine 2481 in a sample (e.g. biological sample) may be accomplished by contacting the sample (e.g. suspected of containing phosphorylated mTOR) with at least one antibody. The antibody is thereby allowed to bind to phosphorylated mTOR at serine 2481 to form an antibody-mTOR complex. The antibody capable of binding to mTOR, or fragment thereof, either free or bound to other mTOR protein subunits, wherein the mTOR is phosphorylated at serine 2481 is also referred to herein as an “anti-S2481 antibody.” The presence of the antibody-mTOR complex is detected using antibody complex detection methods generally known in the art. The presence of the antibody-mTOR complex indicates the presence of phosphorylated mTOR at serine 2481 in the sample.

In some embodiments, the anti-S2481 antibody specifically binds to an mTOR protein, or fragment thereof, that includes a phosphorylated serine at the 2481 position. Thus, the anti-S2481 antibody may preferentially bind to an mTOR protein that includes a phosphorylated S2481 with a dissociation constant (K_{D}) that is lower than an mTOR protein that does not include a phosphorylated S2481. In some embodiments, the dissociation constant (K_{D}) of the anti-S2481 antibody is about 1, 2, 3, 4, 5, 6, 7, 8, 9, 10, 50, 100, or 1000 fold lower (or more) for the mTOR protein that includes a phosphorylated S2481 as compared to the dissociation constant (K_{D}) for the mTOR protein that does not include a phosphorylated S2481.

Biological samples may be obtained from subjects suspected of having a disease involving altered mTOR expression or activity (e.g., lymphoma, glioma, colon cancer, lung cancer, and ovarian cancer). Samples may be analyzed to monitor subjects who have been previously diagnosed as having cancer, to screen subjects who have not been previously diagnosed as carrying cancer, or to monitor the desirability or efficacy of therapeutics targeted at mTOR. Subjects may be either children or adults. In the case of colon cancer, for example, the subjects will most frequently be adult males.

In another embodiment, the invention provides a method for profiling mTOR activation in a test tissue suspected of involving altered mTOR activity, by (a) contacting the test tissue with at least one antibody that binds to a mTOR regulatory subunit only when phosphorylated at S2481 under conditions suitable for formation of an antibody-mTOR complex, (b) detecting the presence of the complex in the test tissue, wherein the presence of the complex indicates the presence of phosphorylated mTOR in the test tissue, and (c) comparing the presence of phosphorylated mTOR detected in step (b) with the presence of phosphorylated mTOR in a control tissue, wherein a difference in mTOR phosphorylation profiles between the test and control tissues indicates altered mTOR activation in the test tissue. In certain embodiments, the antibody is an anti-S2481 antibody. In other embodiments, the test tissue is a cancer tissue, such as breast cancer tissue, lung cancer tissue, lymphoma, glioma, or colon cancer tissue (e.g. suspected of involving altered mTOR phosphorylation).

The methods described above are applicable to examining tissues or samples from mTOR related cancers, such as renal cell carcinoma, colorectal cancer, colon, renal, gastric, liver, pancreatic, esophageal, nasopharyngeal carcinoma, oral, head and neck, lung, hepatocellular, thyroid, acute myeloid leukemia, lymphoma, squamous cell carcinoma (HNSCC), breast cancer, gliomas, glioblastoma, gliosarcoma, and ovarian cancer, endometrial cancer, cervical, uterine, ovarian, melanoma, hepatocellular, astrocytoma, lymphoid, Barrett's adenocarcinomas, in which phosphorylation of mTOR at any of the novel sites disclosed herein has predictive value as to the outcome of the disease or the response of the disease to therapy. It is anticipated that the mTOR antibodies will have diagnostic utility in a disease characterized by, or involving, altered mTOR activity or altered mTOR phosphorylation. The methods are applicable, for example, where samples are taken from a subject that has not been previously diagnosed as having cancer (e.g. lung cancer, breast cancer, lymphoma, glioma, and colon cancer, nor has yet undergone treatment for lung cancer, breast cancer, lymphoma, glioma, or colon cancer). The method is employed to help diagnose the disease, monitor the possible progression of the cancer, or assess risk of the subject developing such cancer involving mTOR phosphorylation. Such diagnostic assay may be carried out prior to preliminary blood evaluation or surgical surveillance procedures.

Such a diagnostic assay may be employed to identify patients with phosphorylated mTOR at serine 2481 (also referred to herein as “activated mTOR”) who would be most likely to respond to cancer therapeutics targeted at inhibiting mTOR activity, such as rapamycin or its analogues (e.g. CCl-779, RADOO1, and AP23573), Torin1, PP30, PP242 and Ku-0063794.

Such a selection of patients would be useful in the clinical evaluation of efficacy of existing or future mTOR inhibitors, as well as in the future prescription of such drugs to patients. Accordingly, in another embodiment, the invention provides a method for selecting a patient suitable for mTOR inhibitor therapy, said method comprising the steps of (a) obtaining at least one biological sample from a patient that is a candidate for mTOR inhibitor therapy, (b) contacting the biological sample with at least one mTOR phospho-specific antibody described herein under conditions suitable for formation of an antibody-mTOR complex, and (c) detecting the presence of the complex in the biological sample, wherein the presence of said complex indicates the presence of phosphorylated mTOR in the test tissue, thereby identifying the patient as potentially suitable for mTOR inhibitor therapy.

Alternatively, the methods are applicable where a subject has been previously diagnosed as having a cancer, e.g. breast cancer, lung cancer, lymphoma, glioma, and colon cancer, and possibly has already undergone treatment for the disease. The method may be employed to monitor the progression of such cancer involving mTOR phosphorylation, or the treatment thereof.

In another embodiment, the invention provides a method for identifying a compound which modulates phosphorylation of mTOR in a test tissue, by (a) contacting the test tissue with the compound, (b) detecting the level of phosphorylated mTOR in the test tissue of step (a) using at least one mTOR phospho-specific antibody described herein under conditions suitable for formation of an antibody-mTOR complex, and (c) comparing the level of phosphorylated mTOR detected in step (b) with the presence of phosphorylated mTOR in a control tissue not contacted with the compound, wherein a difference in mTOR phosphorylation levels between the test and control tissues identifies the compound as a modulator of mTOR phosphorylation. In some preferred embodiments, the test tissue is derived from a subject suspected of having cancer and the compound is a mTOR inhibitor. The compound may modulate mTOR activity either positively or negatively, for example by increasing or decreasing phosphorylation or expression of mTOR. mTOR phosphorylation and activity may be monitored, for example, to determine the efficacy of an anti-mTOR therapeutic, e.g. a mTOR inhibitor.

Conditions suitable for the formation of antibody-antigen complexes or reagent-mTOR complexes are well known in the art. It will be understood that more than one mTOR antibody may be used in the practice of the above-described methods.

### III. Immunoassay Formats & Diagnostic Kits

Assays carried out in accordance with methods of the present invention may be homogeneous assays or heterogeneous assays. In certain homogeneous assays the immunological reaction may involve a mTOR-specific antibody (e.g. an anti-S2481 antibody as described herein), a labeled analyte, and the sample of interest. The signal arising from the label is modified, directly or indirectly, upon the binding of the antibody to the labeled analyte. Both the immunological reaction and detection of the extent thereof are carried out in a homogeneous solution. Immunochemical labels that may be employed include free radicals, radioisotopes, fluorescent dyes, enzymes, bacteriophages, coenzymes, and so forth.

In certain heterogeneous assay approaches, the reagents are usually the sample (e.g. a specimen), a mTOR-specific reagent (e.g., an anti-S2481 antibody), and suitable means for producing a detectable signal. Similar sample (e.g. specimens) as described above may be used. The antibody may be immobilized on a support, such as a bead, plate or slide, and contacted with the specimen suspected of containing the antigen in a liquid phase. The support is then separated from the liquid phase and either the support phase or the liquid phase is examined for a detectable signal employing means for producing such signal. The signal is related to the presence of the analyte in the specimen. Means for producing a detectable signal include the use of radioactive labels, fluorescent labels, enzyme labels, and so forth. For example, if the antigen (e.g. mTOR having a phosphorylated S2481) to be detected contains a second binding site, an antibody which binds to that site can be conjugated to a detectable group and added to the liquid phase reaction solution before the separation step. The presence of the detectable group on the solid support indicates the presence of the antigen in the test sample. Examples of suitable immunoassays are the radioimmunoassay, immunofluorescence methods, enzyme-linked immunoassays, and the like.

Immunoassay formats and variations thereof that may be useful for carrying out the methods disclosed herein are well known in the art. See generally E. Maggio, Enzyme-Immunoassay, (1980) (CRC Press, Inc., Boca Raton, Fla.); see also, e.g., U.S. Pat. No. 4,727,022 (Skold et al, “Methods for Modulating Ligand-Receptor Interactions and their Application”); U.S. Pat. No. 4,659,678 (Forrest et al, “Immunoassay of Antigens”); U.S. Pat. No. 4,376,110 (David et al, “Immunometric Assays Using Monoclonal Antibodies”). Conditions suitable for the formation of reagent-antibody complexes are well described. See id. Monoclonal antibodies of the invention may be used in a “two-site” or “sandwich” assay, with a single cell line serving as a source for both the labeled monoclonal antibody and the bound monoclonal antibody. Such assays are described in U.S. Pat. No. 4,376,110. The concentration of detectable reagent should be sufficient such that the binding of phosphorylated mTOR is detectable compared to background.

mTOR antibodies (e.g. anti-S2481 antibodies) disclosed herein may be conjugated to a solid support suitable for a diagnostic assay (e.g., beads, plates, slides or wells formed from materials such as latex or polystyrene) in accordance with known techniques, such as precipitation. Antibodies may likewise be conjugated to detectable groups such as radiolabels (e.g., ³⁵S, ¹²⁵I, ¹³¹I), enzyme labels (e.g., horseradish peroxidase, alkaline phosphatase), Quantum dots (Qdots) and fluorescent labels (e.g., fluorescein) in accordance with known techniques. mTOR antibodies may also be optimized for use in a flow cytometry assay to determine the activation status of mTOR in patients before, during, and after treatment with a drug targeted at inhibiting mTOR phosphorylation at a serine site disclosed herein. For example, bone marrow cells or peripheral blood cells from patients may be analyzed by flow cytometry for mTOR phosphorylation, as well as for markers identifying various hematopoietic cell types. In this manner, mTOR activation status (e.g. mTOR phosphorylated at S2481) of the malignant cells may be specifically characterized.

Flow cytometry may be carried out according to standard methods. See, e.g. Chow et al, Cytometry (Communications in Clinical Cytometry) 46:72-78 (2001). Briefly and by way of example, the following protocol for cytometric analysis may be employed: fixation of the cells with 1% paraformaldehyde for 10 minutes at 370C followed by permeabilization in 90% methanol for 30 minutes on ice. Cells may then be stained with the primary mTOR antibody, washed and labeled with a fluorescent-labeled secondary antibody. Alternatively, the cells may be stained with a fluorescent-labeled primary antibody. The cells would then be analyzed on a flow cytometer {e.g. a Beckman Coulter EPICS-XL) according to the specific protocols of the instrument used. Such an analysis would identify the presence of activated mTOR in the malignant cells and reveal the drug response on the targeted mTOR protein. Alternatively, mTOR antibodies may be optimized for use in other clinically-suitable applications, for example bead-based multiplex-type assays, such as IGEN, Luminex™ and/or Bioplex™ assay formats, or otherwise optimized for antibody arrays formats.

The use of the antibodies in a RIA assay are additionally contemplated. The radioimmunoassay (RIA) is an analytical technique which depends on the competition (affinity) of an antigen for antigen-binding sites on antibody molecules. Standard curves are constructed from data gathered from a series of samples each containing the same known concentration of labeled antigen, and various, but known, concentrations of unlabeled antigen. Antigens are labeled with a radioactive isotope tracer. The mixture is incubated in contact with an antibody. Then the free antigen is separated from the antibody and the antigen bound thereto. Then, by use of a suitable detector, such as a gamma or beta radiation detector, the percent of either the bound or free labeled antigen or both is determined. This procedure is repeated for a number of samples containing various known concentrations of unlabeled antigens and the results are plotted as a standard graph. The percent of bound tracer antigens is plotted as a function of the antigen concentration. Typically, as the total antigen concentration increases the relative amount of the tracer antigen bound to the antibody decreases. After the standard graph is prepared, it is thereafter used to determine the concentration of antigen in samples undergoing analysis.

In an analysis, the sample in which the concentration of antigen is to be determined is mixed with a known amount of tracer antigen. Tracer antigen is the same antigen known to be in the sample but which has been labeled with a suitable radioactive isotope. The sample with tracer is then incubated in contact with the antibody. Then it can be counted in a suitable detector which counts the free antigen remaining in the sample. The antigen (e.g. mTOR phosphorylated at S2481) bound to the antibody or immunoadsorbent may also be similarly counted. Then, from the standard curve, the concentration of antigen in the original sample is determined.

Diagnostic kits for carrying out the methods disclosed above are also provided by the invention. Such kits comprise at least one detectable reagent that binds to mTOR when phosphorylated at the serine phosphorylation site disclosed herein (S2481) (e.g. an anti-S2481 antibody). In one embodiment, the diagnostic kit comprises (a) a mTOR antibody (e.g. an anti-S2481 antibody) conjugated to a solid support and (b) a second antibody conjugated to a detectable group. The reagents may also include ancillary agents such as buffering agents and protein stabilizing agents, e.g., polysaccharides and the like. The diagnostic kit may further include, where necessary, other members of the signal-producing system of which system the detectable group is a member (e.g., enzyme substrates), agents for reducing background interference in a test, control reagents, apparatus for conducting a test, and the like. In another embodiment, a kit (e.g. a kit for the selection of a patient suitable for mTOR inhibitor therapy) comprises (a) a mTOR antibody (e.g. an anti-S2481 antibody), and (b) a specific binding partner (i.e. secondary antibody) conjugated to a detectable group.

The primary (phospho-mTOR) detection antibody may itself be directly labeled with a detectable group, or alternatively, a secondary antibody, itself labeled with a detectable group, that binds to the primary antibody may be employed. Labels (including dyes and the like) suitable as detectable agents are well known in the art. Ancillary agents as described above may likewise be included. The test kit may be packaged in any suitable manner, typically with all elements in a single container along with a sheet of printed instructions for carrying out the test.

In reference to antibody detection methods, “detection reagents” are meant reagents that can be used to detect antibodies, including both primary or secondary antibodies. For example, detection reagents can be fluorescent detect ion reagents, qdots, chromogenic detect ion reagents, or polymer based detect ion systems. However, the methods and kits of the invention are not limited by these detect ion reagents, nor are they limited to a primary and secondary antibody scheme (for example, tertiary, etc. antibodies are contemplated by the methods of the invention).

Gene-specific probes may be designed according to any of the following published procedures. To this end it is important to produce pure, or homogeneous, probes to minimize hybridizations at locations other than at the site of interest (Henderson, 1982, International Review of Cytology, 76:1-46). Manuelidis et al, Chromosoma, 91:28-38 (1984), discloses the construction of a single kind of DNA probe for detecting multiple loci on chromosomes corresponding to members of a family of repeated DNA sequences. Wallace et al., Nucleic Acids Research, 9:879-94 (1981), discloses the construction of synthetic oligonucleotide probes having mixed base sequences for detecting a single locus corresponding to a structural gene. The mixture of base sequences was determined by considering all possible nucleotide sequences that could code for a selected sequence of amino acids in the protein to which the structural gene corresponded. Olsen et al., Biochemistry, 19:2419-28 (1980), discloses a method for isolating labeled unique sequence human X chromosomal DNA by successive hybridizations: first, total genomic human DNA against itself so that a unique sequence DNA fraction can be isolated; second, the isolated unique sequence human DNA fraction against mouse DNA so that homologous mouse/human sequences are removed; and finally, the unique sequence human DNA not homologous to mouse against the total genomic DNA of a human/mouse hybrid whose only human chromosome is chromosome X, so that a fraction of unique sequence X chromosomal DNA is isolated.

### IV. Antibodies and Cell Lines

As described above, an anti-S2481 antibody may bind to an mTOR or fragment thereof when phosphorylated at S2481, but do not substantially bind to mTOR when not phosphorylated at this respective site, nor to mTOR when phosphorylated at other residues. The mTOR antibodies (e.g. anti-S2481 antibody) provided herein include (a) monoclonal antibody which binds phospho-mTOR sites described above, (b) polyclonal antibodies which bind to phospho-mTOR sites described above, (c) antibodies (monoclonal or polyclonal) which specifically bind to the phospho-antigen (e.g. phosphorylated S2481) (or the epitope) bound by the exemplary mTOR phospho-specific antibodies disclosed herein, and (d) fragments of (a), (b), or (c) above which bind to the antigen (e.g. mTOR phosphorylated at S2481). Such antibodies and antibody fragments may be produced by a variety of techniques well known in the art, as discussed below. Antibodies that bind to the phosphorylated epitope (i.e., the specific binding site) bound by the exemplary mTOR antibodies described herein can be identified in accordance with known techniques, such as their ability to compete with labeled mTOR antibodies in a competitive binding assay. The epitopic site of the mTOR antibodies of the invention may be a region lying between the catalytic domain and the FATC domain near the C-terminus of mTOR, more preferably a peptide fragment consisting essentially of approximately 20 amino acids comprising residues 2471-2491, which contains S2481.

The methods provided herein may also use molecules equivalent to mTOR antibodies such as protein binding domains or nucleic acid aptamers, which bind, in a phospho-specific manner, to essentially the same phosphorylated epitope to which the mTOR antibodies described above bind. See, e.g., Neuberger et al, Nature, 312:604 (1984). Such equivalent non-antibody reagents may be suitably employed in the methods of the invention further described herein.

The term “antibody” or “antibodies” as used herein refers to all types of immunoglobulins, including IgG, IgM, IgA, IgD, and IgE, and any sub-isotype, including IgG1, IgG2a, IgG2b, IgG3 and IgG4, IgE1, IgE2 etc., and may include including Fab or antigen-recognition fragments thereof. The antibodies may be monoclonal or polyclonal and may be of any species of origin, including (for example) mouse, rat, rabbit, horse, or human, or may be chimeric antibodies. See, e.g., M. Walker et al., Molec. Immunol. 26:403-11 (1989); Morrision et al., Proc. Nat'l. Acad. Sci., 81:6851 (1984); Neuberger et al, Nature, 312:604 (1984)). The antibodies may be recombinant monoclonal antibodies produced according to the methods disclosed in U.S. Pat. No. 4,474,893 (Reading) or U.S. Pat. No. 4,816,567 (Cabilly et al.). The antibodies may also be chemically constructed by specific antibodies made according to the method disclosed in U.S. Pat. No. 4,676,980 (Segel et al).

The chimeric antibody is an antibody having portions derived from different antibodies. For example, a chimeric antibody may have a variable region and a constant region derived from two different antibodies. The donor antibodies may be from different species. In certain embodiments, the variable region of a chimeric antibody is non-human, e.g., murine, and the constant region is human.

“Genetically altered antibodies” refer to antibodies wherein the amino acid sequence has been varied from that of a native antibody. Because of the relevance of recombinant DNA techniques to this application, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The term “mTOR antibodies” includes phospho-specific antibodies that selectively bind mTOR regulatory subunit only when phosphorylated at a serine phosphorylation site S2481 in mTOR, both monoclonal and polyclonal, as disclosed herein.

The term “does not bind” with respect to such antibodies means does not substantially react with as compared to binding to phospho-mTOR. The antibodies may bind the regulatory subunit alone or when complexed with Raptor, mLST8 and PRAS40 to form the complete mTor C1 complex or Rictor, mSin1, mLST8 and Protor to form the complete mTorC2 complex. In some embodiments, the antibody may bind the regulatory subunit alone or when complexed with Rictor, mSin1, mLST8 and Protor to form the complete mTorC2 complex. The term “does not bind”, when appeared in context of an antibody's binding to one phospho-form (e.g., phosphorylated form) of a sequence, means that the antibody does not substantially react with the other phospho-form (e.g., non-phosphorylated form) of the same sequence. One of skill in the art will appreciate that the expression may be applicable in those instances when (1) a phospho-specific antibody either does not apparently bind to the non-phospho form of the antigen as ascertained in commonly used experimental detection systems (Western blotting, IHC, Immunofluorescence, etc.); (2) where there is some reactivity with the surrounding amino acid sequence, but that the phosphorylated residue is an immunodominant feature of the reaction. In cases such as these, there is an apparent difference in affinities for the two sequences. Dilutional analyses of such antibodies indicates that the antibodies apparent affinity for the phosphorylated form is at least 10-100 fold higher than for the non-phosphorylated form; or where (3) the phospho-specific antibody reacts no more than an appropriate control antibody would react under identical experimental conditions. A control antibody preparation might be, for instance, purified immunoglobulin from a pre-immune animal of the same species, an isotype- and species-matched monoclonal antibody. Tests using control antibodies to demonstrate specificity are recognized by one of skill in the art as appropriate and definitive. The term “detectable reagent” means a molecule, including an antibody, peptide fragment, binding protein domain, etc., the binding of which to a desired target is detectable or traceable. Suitable means of detection are described below. In particular embodiments, the antibodies of the present application are attached to labeling moieties, such as a detectable marker. One or more detectable labels can be attached to the antibodies. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

Polyclonal antibodies of the invention may be produced according to standard techniques by immunizing a suitable animal (e.g., rabbit, goat, etc.) with an antigen encompassing a mTOR phosphorylation site described herein, collecting immune serum from the animal, and separating the polyclonal antibodies from the immune serum, in accordance with known procedures. In a preferred embodiment, the antigen is a phospho-peptide antigen comprising the site sequence surrounding and including the respective phosphorylated serine residue described herein, the antigen being selected and constructed in accordance with well-known techniques. See, e.g., ANTIBODIES: A LABORATORY MANUAL, Chapter 5, p. 75-76, Harlow & Lane Eds., Cold Spring Harbor Laboratory (1988); Czernik, Methods In Enzymology, 201:264-283 (1991); Merrifield, J. Am. Chem. Soc., 85:21-49 (1962)). An exemplary peptide antigen, gttypesih(phospho)-sFigdglvkp for mTOR is described in the Examples, below. It will be appreciated by those of skill in the art that longer or shorter phosphopeptide antigens may be employed. As used herein, the term “epitope” refers to the smallest portion of a protein capable of selectively binding to the antigen binding site of an antibody. It is well accepted by those skilled in the art that the minimal size of a protein epitope capable of selectively binding to the antigen binding site of an antibody is about five or six to seven amino acids. See Id. Polyclonal mTOR antibodies produced as described herein may be screened as further described below. Monoclonal antibodies of the invention may be produced in a hybridoma cell line according to the well-known technique of Kohler and Milstein, Nature 265: 495-97 (1975); Kohler and Milstein, Eur. J. Immunol., 6:511 (1976); see also, CURRENT PROTOCOLS IN MOLECULAR BIOLOGY, Ausubel et al., Eds. (1989). Monoclonal antibodies so produced are highly specific, and improve the selectivity and specificity of diagnostic assay methods provided by the invention.

For example, a solution containing the appropriate antigen may be injected into a mouse or other species and, after a sufficient time (in keeping with conventional techniques), the animal is sacrificed and spleen cells obtained. The spleen cells are then immortalized by fusing them with myeloma cells, typically in the presence of polyethylene glycol, to produce hybridoma cells. Rabbit fusion hybridomas, for example, may be produced as described in U.S. Pat. No. 5,675,063, C. Knight, Issued Oct. 7, 1997. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art. See, e.g., W. Huse, Science, 246:1275-81 (1989); Mullinax et al., Proc. Nat. 7 Acad. Sci., 87:8095 (1990). If monoclonal antibodies of one isotype are preferred for a particular application, particular isotypes can be prepared directly, by selecting from the initial fusion, or prepared secondarily, from a parental hybridoma secreting a monoclonal antibody of different isotype by using the sib selection technique to isolate class-switch variants (Steplewski, et al., Proc. Nat.'l. Acad. Sci, 82:8653 (1985); Spira et al, J. Immunol. Methods, 74:307 (1984)).

Monoclonal antibodies of the invention may be produced recombinantly by expressing the encoding nucleic acids in a suitable host cell under suitable conditions. Accordingly, the invention further provides host cells comprising the nucleic acids and vectors described herein.

Other antibodies specifically contemplated are oligoclonal antibodies. As used herein, the phrase “oligoclonal antibodies” refers to a predetermined mixture of distinct monoclonal antibodies. See, e.g., PCT publication WO 95/20401; U.S. Pat. Nos. 5,789,208 and 6,335,163. In one embodiment, oligoclonal antibodies consisting of a predetermined mixture of antibodies against one or more epitopes are generated in a single cell. In other embodiments, oligoclonal antibodies comprise a plurality of heavy chains capable of pairing with a common light chain to generate antibodies with multiple specificities (e.g., PCT publication WO 04/009618). Oligoclonal antibodies are particularly useful when it is desired to target multiple epitopes on a single target molecule. In view of the assays and epitopes disclosed herein, those skilled in the art can generate or select antibodies or mixtures of antibodies that are applicable for an intended purpose and desired need.

Recombinant antibodies against the phosphorylation sites identified in the invention are also included in the present application. These recombinant antibodies have the same amino acid sequence as the natural antibodies or have altered amino acid sequences of the natural antibodies in the present application. They can be made in any expression systems including both prokaryotic and eukaryotic expression systems or using phage display methods (see, e.g., Dower et al., WO91/17271 and McCafferty et al., WO92/01047; U.S. Pat. No. 5,969,108, which are herein incorporated by reference in their entirety).

Antibodies can be engineered in numerous ways. They can be made as single-chain antibodies (including small modular immunopharmaceuticals or SMIPs™), Fab and F(ab′)2 fragments, etc. Antibodies can be humanized, chimerized, deimmunized, or fully human. Numerous publications set forth the many types of antibodies and the methods of engineering such antibodies. For example, see U.S. Pat. Nos. 6,355,245; 6,180,370; 5,693,762; 6,407,213; 6,548,640; 5,565,332; 5,225,539; 6,103,889; and 5,260,203.

The genetically altered antibodies should be functionally equivalent to the above-mentioned natural antibodies. In certain embodiments, modified antibodies provide improved stability or/and therapeutic efficacy. Examples of modified antibodies include those with conservative substitutions of amino acid residues, and one or more deletions or additions of amino acids that do not significantly deleteriously alter the antigen binding utility. Substitutions can range from changing or modifying one or more amino acid residues to complete redesign of a region as long as the therapeutic utility is maintained. Antibodies of this application can be modified post-translationally (e.g., acetylation, and/or phosphorylation) or can be modified synthetically (e.g., the attachment of a labeling group). The genetically altered antibodies used in the invention include CDR grafted humanized antibodies. In one embodiment, the humanized antibody comprises heavy and/or light chain CDRs of a non-human donor immunoglobulin and heavy chain and light chain frameworks and constant regions of a human acceptor immunoglobulin. The method of making humanized antibody is disclosed in U.S. Pat. Nos. 5,530,101; 5,585,089; 5,693,761; 5,693,762; and 6,180,370 each of which is incorporated herein by reference in its entirety.

Antibodies with engineered or variant constant or Fc regions can be useful in modulating effector functions, such as, for example, antigen-dependent cytotoxicity (ADCC) and complement-dependent cytotoxicity (CDC). Such antibodies with engineered or variant constant or Fc regions may be useful in instances where a parent singling protein (Table 1) is expressed in normal tissue; variant antibodies without effector function in these instances may elicit the desired therapeutic response while not damaging normal tissue. Accordingly, certain aspects and methods of the present disclosure relate to antibodies with altered effector functions that comprise one or more amino acid substitutions, insertions, and/or deletions.

Antigen-binding fragments of the antibodies of the invention, which retain the binding specificity of the intact antibody, are also included in the invention. Examples of these antigen-binding fragments include, but are not limited to, partial or full heavy chains or light chains, variable regions, or CDR regions of any phosphorylation site-specific antibodies described herein.

In some instances the antibody fragments are truncated chains (truncated at the carboxyl end). In certain embodiments, these truncated chains possess one or more immunoglobulin activities (e.g., complement fixation activity). Examples of truncated chains include, but are not limited to, Fab fragments (consisting of the VL, VH, CL and CH1 domains); Fd fragments (consisting of the VH and CH1 domains); Fv fragments (consisting of VL and VH domains of a single chain of an antibody); dAb fragments (consisting of a VH domain); isolated CDR regions; (Fab′)2 fragments, bivalent fragments (comprising two Fab fragments linked by a disulphide bridge at the hinge region). The truncated chains can be produced by conventional biochemical techniques, such as enzyme cleavage, or recombinant DNA techniques, each of which is known in the art. These polypeptide fragments may be produced by proteolytic cleavage of intact antibodies by methods well known in the art, or by inserting stop codons at the desired locations in the vectors using site-directed mutagenesis, such as after CH1 to produce Fab fragments or after the hinge region to produce (Fab′)2 fragments. Single chain antibodies may be produced by joining VL- and VH-coding regions with a DNA that encodes a peptide linker connecting the VL and VH protein fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment of an antibody yields an F(ab′)2 fragment that has two antigen-combining sites and is still capable of cross-linking antigen. “Fv” usually refers to the minimum antibody fragment that contains a complete antigen-recognition and -binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the CDRs confer antigen-binding specificity to the antibody. However, even a single variable domain (or half of an Fv comprising three CDRs specific for an antigen) has the ability to recognize and bind antigen, although likely at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

“Single-chain Fv” or “scFv” antibody fragments comprise the VH and VL domains of an antibody, wherein these domains are present in a single polypeptide chain. In certain embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore, eds. (Springer-Verlag: New York, 1994), pp. 269-315. Bispecific antibodies may be monoclonal, human or humanized antibodies that have binding specificities for at least two different antigens. In the present case, one of the binding specificities is for the phosphorylation site, the other one is for any other antigen, such as for example, a cell-surface protein or receptor or receptor subunit. Alternatively, a therapeutic agent may be placed on one arm. The therapeutic agent can be a drug, toxin, enzyme, DNA, radionuclide, etc.

In some instances, the antigen-binding fragment can be a diabody. The term “diabody” refers to small antibody fragments with two antigen-binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993).

Camelid antibodies refer to a unique type of antibodies that are devoid of light chain, initially discovered from animals of the camelid family. The heavy chains of these so-called heavy-chain antibodies bind their antigen by one single domain, the variable domain of the heavy immunoglobulin chain, referred to as VHH. VHHs show homology with the variable domain of heavy chains of the human VHIII family. The VHHs obtained from an immunized camel, dromedary, or llama have a number of advantages, such as effective production in microorganisms such as Saccharomyces cerevisiae.

In certain embodiments, single chain antibodies, and chimeric, humanized or primatized (CDR-grafted) antibodies, as well as chimeric or CDR-grafted single chain antibodies, comprising portions derived from different species, are also encompassed by the present disclosure as antigen-binding fragments of an antibody. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., U.S. Pat. Nos. 4,816,567 and 6,331,415; U.S. Pat. No. 4,816,397; European Patent No. 0,120,694; WO 86/01533; European Patent No. 0,194,276 B1; U.S. Pat. No. 5,225,539; and European Patent No. 0,239,400 B1. See also, Newman et al., BioTechnology, 10:1455-1460 (1992), regarding primatized antibody. See, e.g., Ladner et al., U.S. Pat. No. 4,946,778; and Bird et al., Science, 242:423-426 (1988)), regarding single chain antibodies.

In addition, functional fragments of antibodies, including fragments of chimeric, humanized, primatized or single chain antibodies, can also be produced. Functional fragments of the subject antibodies retain at least one binding function and/or modulation function of the full-length antibody from which they are derived.

Also contemplated are other equivalent non-antibody molecules, such as protein binding domains or aptamers, which bind, in a phospho-specific manner, to an amino acid sequence comprising a novel phosphorylation site of the invention. See, e.g., Neuberger et al, Nature, 312:604 (1984). Aptamers are oligonucleic acid or peptide molecules that bind a specific target molecule. DNA or RNA aptamers are typically short oligonucleotides, engineered through repeated rounds of selection to bind to a molecular target. Peptide aptamers typically consist of a variable peptide loop attached at both ends to a protein scaffold. This double structural constraint generally increases the binding affinity of the peptide aptamer to levels comparable to an antibody (nanomolar range).

The invention also provides hybridoma clones, constructed as described above, that produce mTOR monoclonal antibodies of the invention. Similarly, the invention includes recombinant cells producing a mTOR antibody as disclosed herein, which cells may be constructed by well known techniques; for example the antigen combining site of the monoclonal antibody can be cloned by PCR and single-chain antibodies produced as phage-displayed recombinant antibodies or soluble antibodies in E. coli {see, e.g., ANTIBODY ENGINEERING PROTOCOLS, 1995, Humana Press, Sudhir Paul editor.)

mTOR antibodies, whether polyclonal or monoclonal, may be screened for epitope and phospho-specificity according to standard techniques. See, e.g. Czernik et al, Methods in Enzymology, 201:264-283 (1991). For example, the antibodies may be screened against the phospho and non-phospho peptide library by ELISA to ensure specificity for both the desired antigen (i.e. that epitope including a serine phosphorylation site disclosed herein) and for reactivity only with the phosphorylated form of the antigen. Peptide competition assays may be carried out to confirm lack of reactivity with other mTOR phospho-epitopes. The antibodies may also be tested by Western blotting against cell preparations containing mTOR, e.g. cell lines over-expressing mTOR, to confirm reactivity with the desired phosphorylated target.

A “phosphorylatable” amino acid refers to an amino acid that is capable of being modified by addition of a phosphate group (any includes both phosphorylated form and unphosphorylated form). Alternatively, the serine may be deleted. Residues other than the serine may also be modified (e.g., delete or mutated) if such modification inhibits the phosphorylation of the serine residue. For example, residues flanking the serine may be deleted or mutated, so that a kinase can not recognize/phosphorylate the mutated protein or the peptide. Standard mutagenesis and molecular cloning techniques can be used to create amino acid substitutions or deletions.

mTOR antibodies may be further characterized via immunohistochemical (IHC) staining using normal and diseased tissues to examine mTOR phosphorylation and activation status in diseased tissue. IHC may be carried out according to well-known techniques. See, e.g., ANTIBODIES: A LABORATORY MANUAL, Chapter 10, Harlow & Lane Eds., Cold Spring Harbor Laboratory (1988). Briefly, paraffin-embedded tissue (e.g. tumor tissue) is prepared for immunohistochemical staining by deparaffinizing tissue sections with xylene followed by ethanol; hydrating in water then PBS; unmasking antigen by heating slide in sodium citrate buffer; incubating sections in hydrogen peroxide; blocking in blocking solution; incubating slide in primary antibody and secondary antibody; and finally detecting using ABC avidin/biotin method according to manufacturer's instructions.

### V. Therapeutic Uses

Also provided are methods and compositions for therapeutic uses of the peptides or proteins comprising a phosphorylation site described herein, and phosphorylation site-specific antibodies described herein. The invention provides for a method of treating or preventing a disease such as, for example, carcinoma in a subject, wherein the carcinoma is associated with the phosphorylation state of a phosphorylation site, whether phosphorylated or dephosphorylated, comprising: administering to a subject in need thereof a therapeutically effective amount of a peptide comprising a phosphorylation site and/or an antibody or antigen-binding fragment thereof that specifically bind The phosphorylation site of the invention. The antibodies maybe full-length antibodies, genetically engineered antibodies, antibody fragments, and antibody conjugates of the invention.

The antibodies of the invention may also be used to target cancer cells for effector-mediated cell death. The antibody disclosed herein may be administered as a fusion molecule that includes a phosphorylation site-targeting portion joined to a cytotoxic moiety to directly kill cancer cells. Alternatively, the antibody may directly kill the cancer cells through complement-mediated or antibody-dependent cellular cytotoxicity. Accordingly in one embodiment, the antibodies of the present disclosure may be used to deliver a variety of cytotoxic compounds. Any cytotoxic compound can be fused to the present antibodies. The fusion can be achieved chemically or genetically (e.g., via expression as a single, fused molecule). The cytotoxic compound can be a biological, such as a polypeptide, or a small molecule. As those skilled in the art will appreciate, for small molecules, chemical fusion is used, while for biological compounds, either chemical or genetic fusion can be used.

Non-limiting examples of cytotoxic compounds include therapeutic drugs, radiotherapeutic agents, ribosome-inactivating proteins (RIPs), chemotherapeutic agents, toxic peptides, toxic proteins, and mixtures thereof. The cytotoxic drugs an be intracellularly acting cytotoxic drugs, such as short-range radiation emitters, including, for example, short-range, high-energy Î±-emitters. Enzymatically active toxins and fragments thereof, including ribosome-inactivating proteins, are exemplified by saporin, luffin, momordins, ricin, trichosanthin, gelonin, abrin, etc. Procedures for preparing enzymatically active polypeptides of the immunotoxins are described in WO84/03508 and WO85/03508, which are hereby incorporated by reference. Certain cytotoxic moieties are derived from adriamycin, chlorambucil, daunomycin, methotrexate, neocarzinostatin, and platinum, for example. Exemplary chemotherapeutic agents that may be attached to an antibody or antigen-binding fragment thereof include taxol, doxorubicin, verapamil, podophyllotoxin, procarbazine, mechlorethamine, cyclophosphamide, camptothecin, ifosfamide, melphalan, chlorambucil, bisulfan, nitrosurea, dactinomycin, daunorubicin, doxorubicin, bleomycin, plicomycin, mitomycin, etoposide (VP16), tamoxifen, transplatinum, 5-fluorouracil, vincristin, vinblastin, or methotrexate.

Procedures for conjugating the antibodies with the cytotoxic agents have been previously described and are within the purview of one skilled in the art. Alternatively, the antibody can be coupled to high energy radiation emitters, for example, a radioisotope, such as 131I, a Î³-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, Baldwin et al. (eds.), pp. 303-316 (Academic Press 1985), which is hereby incorporated by reference. Other suitable radioisotopes include Î±-emitters, such as 212Bi, 213Bi, and 211At, and Î²-emitters, such as 186Re and 90Y.

Because many of the signaling proteins in which novel serine phosphorylation sites of the invention occur also are expressed in normal cells and tissues, it may also be advantageous to administer a phosphorylation site-specific antibody with a constant region modified to reduce or eliminate ADCC or CDC to limit damage to normal cells. For example, effector function of an antibodies may be reduced or eliminated by utilizing an IgG1 constant domain instead of an IgG2/4 fusion domain. Other ways of eliminating effector function can be envisioned such as, e.g., mutation of the sites known to interact with FcR or insertion of a peptide in the hinge region, thereby eliminating critical sites required for FcR interaction. Variant antibodies with reduced or no effector function also include variants as described previously herein.

The peptides and antibodies of the invention may be used in combination with other therapies or with other agents. Other agents include but are not limited to polypeptides, small molecules, chemicals, metals, organometallic compounds, inorganic compounds, nucleic acid molecules, oligonucleotides, aptamers, spiegelmers, antisense nucleic acids, locked nucleic acid (LNA) inhibitors, peptide nucleic acid (PNA) inhibitors, immunomodulatory agents, antigen-binding fragments, prodrugs, and peptidomimetic compounds. In certain embodiments, the antibodies and peptides of the invention may be used in combination with cancer therapies known to one of skill in the art.

In certain aspects, the present disclosure relates to combination treatments comprising a phosphorylation site-specific antibody described herein and immunomodulatory compounds, vaccines or chemotherapy. Illustrative examples of suitable immunomodulatory agents that may be used in such combination therapies include agents that block negative regulation of T cells or antigen presenting cells (e.g., anti-CTLA4 antibodies, anti-PD-L1 antibodies, anti-PDL-2 antibodies, anti-PD-1 antibodies and the like) or agents that enhance positive co-stimulation of T cells (e.g., anti-CD40 antibodies or anti 4-IBB antibodies) or agents that increase NK cell number or T-cell activity (e.g., inhibitors such as IMiDs, thalidomide, or thalidomide analogs). Furthermore, immunomodulatory therapy could include cancer vaccines such as dendritic cells loaded with tumor cells, proteins, peptides, RNA, or DNA derived from such cells, patient derived heat-shock proteins (hsp's) or general adjuvants stimulating the immune system at various levels such as CpG, Luivac®, Biostim®, Ribomunyl®, Imudon®, Broncho Vaxom® or any other compound or other adjuvant activating receptors of the innate immune system (e.g., toll like receptor agonist, anti-CTLA-4 antibodies, etc.). Also, immunomodulatory therapy could include treatment with cytokines such as IL-2, GM-CSF and IFN-gamma.

Furthermore, combination of antibody therapy with chemotherapeutics could be particularly useful to reduce overall tumor burden, to limit angiogenesis, to enhance tumor accessibility, to enhance susceptibility to ADCC, to result in increased immune function by providing more tumor antigen, or to increase the expression of the T cell attractant LIGHT.

Pharmaceutical compounds that may be used for combinatory anti-tumor therapy include, merely to illustrate: aminoglutethimide, amsacrine, anastrozole, asparaginase, beg, bicalutamide, bleomycin, buserelin, busulfan, camptothecin, capecitabine, carboplatin, carmustine, chlorambucil, cisplatin, cladribine, clodronate, colchicine, cyclophosphamide, cyproterone, cytarabine, dacarbazine, dactinomycin, daunorubicin, dienestrol, diethylstilbestrol, docetaxel, doxorubicin, epirubicin, estradiol, estramustine, etoposide, exemestane, filgrastim, fludarabine, fludrocortisone, fluorouracil, fluoxymesterone, flutamide, gemcitabine, genistein, goserelin, hydroxyurea, idarubicin, ifosfamide, imatinib, interferon, irinotecan, letrozole, leucovorin, leuprolide, levamisole, lomustine, mechlorethamine, medroxyprogesterone, megestrol, melphalan, mercaptopurine, mesna, methotrexate, mitomycin, mitotane, mitoxantrone, nilutamide, nocodazole, octreotide, oxaliplatin, paclitaxel, pamidronate, pentostatin, plicamycin, porfimer, procarbazine, raltitrexed, rituximab, streptozocin, suramin, tamoxifen, temozolomide, teniposide, testosterone, thioguanine, thiotepa, titanocene dichloride, topotecan, trastuzumab, tretinoin, vinblastine, vincristine, vindesine, and vinorelbine.

These chemotherapeutic anti-tumor compounds may be categorized by their mechanism of action into groups, including, for example, the following classes of agents: anti-metabolites/anti-cancer agents, such as pyrimidine analogs (5-fluorouracil, floxuridine, capecitabine, gemcitabine and cytarabine) and purine analogs, folate inhibitors and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristine, vinblastine, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, Cytoxan, dactinomycin, daunorubicin, doxorubicin, epirubicin, hexamethylmelamineoxaliplatin, iphosphamide, melphalan, mechlorethamine, mitomycin, mitoxantrone, nitrosourea, plicamycin, procarbazine, taxol, taxotere, teniposide, triethylenethiophosphoramide and etoposide (VP 16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamy cin), azathioprine, mycophenolate mofetil); immunomodulatory agents (thalidomide and analogs thereof such as lenalidomide (Revlimid, CC-5013) and CC-4047 (Actimid)), cyclophosphamide; anti-angiogenic compounds (TNP-470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin and mitoxantrone, topotecan, irinotecan), corticosteroids (cortisone, dexamethasone, hydrocortisone, methylprednisolone, prednisone, and prenisolone); growth factor signal transduction kinase inhibitors; mitochondrial dysfunction inducers and caspase activators; and chromatin disruptors.

In certain embodiments, pharmaceutical compounds that may be used for combinatory anti-angiogenesis therapy include: (1) inhibitors of release of “angiogenic molecules,” such as bFGF (basic fibroblast growth factor); (2) neutralizers of angiogenic molecules, such as anti-Î²bFGF antibodies; and (3) inhibitors of endothelial cell response to angiogenic stimuli, including collagenase inhibitor, basement membrane turnover inhibitors, angiostatic steroids, fungal-derived angiogenesis inhibitors, platelet factor 4, thrombospondin, arthritis drugs such as D-penicillamine and gold thiomalate, vitamin D3 analogs, alpha-interferon, and the like. For additional proposed inhibitors of angiogenesis, see Blood et al., Biochim. Biophys. Acta, 1032:89-118 (1990), Moses et al., Science, 248:1408-1410 (1990), Ingber et al., Lab. Invest., 59:44-51 (1988), and U.S. Pat. Nos. 5,092,885, 5,112,946, 5,192,744, 5,202,352, and 6,573,256. In addition, there are a wide variety of compounds that can be used to inhibit angiogenesis, for example, peptides or agents that block the VEGF-mediated angiogenesis pathway, endostatin protein or derivatives, lysine binding fragments of angiostatin, melanin or melanin-promoting compounds, plasminogen fragments (e.g., Kringles 1-3 of plasminogen), troponin subunits, inhibitors of vitronectin Î±vÎ²3, peptides derived from Saposin B, antibiotics or analogs (e.g., tetracycline or neomycin), dienogest-containing compositions, compounds comprising a MetAP-2 inhibitory core coupled to a peptide, the compound EM-138, chalcone and its analogs, and naaladase inhibitors. See, for example, U.S. Pat. Nos. 6,395,718, 6,462,075, 6,465,431, 6,475,784, 6,482,802, 6,482,810, 6,500,431, 6,500,924, 6,518,298, 6,521,439, 6,525,019, 6,538,103, 6,544,758, 6,544,947, 6,548,477, 6,559,126, and 6,569,845.

### VI. Pharmaceutical Formulations and Methods of Administration

Methods of administration of therapeutic agents, particularly peptide and antibody therapeutics, are well-known to those of skill in the art.

Peptides of the invention can be administered in the same manner as conventional peptide type pharmaceuticals. Preferably, peptides are administered parenterally, for example, intravenously, intramuscularly, intraperitoneally, or subcutaneously. When administered orally, peptides may be proteolytically hydrolyzed. Therefore, oral application may not be usually effective. However, peptides can be administered orally as a formulation wherein peptides are not easily hydrolyzed in a digestive tract, such as liposome-microcapsules. Peptides may be also administered in suppositories, sublingual tablets, or intranasal spray. If administered parenterally, a preferred pharmaceutical composition is an aqueous solution that, in addition to a peptide of the invention as an active ingredient, may contain for example, buffers such as phosphate, acetate, etc., osmotic pressure-adjusting agents such as sodium chloride, sucrose, and sorbitol, etc., antioxidative or antioxy genie agents, such as ascorbic acid or tocopherol and preservatives, such as antibiotics. The parenterally administered composition also may be a solution readily usable or in a lyophilized form which is dissolved in sterile water before administration.

The pharmaceutical formulations, dosage forms, and uses described below generally apply to antibody-based therapeutic agents, but are also useful and can be modified, where necessary, for making and using therapeutic agents of the disclosure that are not antibodies.

To achieve the desired therapeutic effect, the phosphorylation site-specific antibodies or antigen-binding fragments thereof can be administered in a variety of unit dosage forms. The dose will vary according to the particular antibody. For example, different antibodies may have different masses and/or affinities, and thus require different dosage levels. Antibodies prepared as Fab or other fragments will also require differing dosages than the equivalent intact immunoglobulins, as they are of considerably smaller mass than intact immunoglobulins, and thus require lower dosages to reach the same molar levels in the patient's blood. The dose will also vary depending on the manner of administration, the particular symptoms of the patient being treated, the overall health, condition, size, and age of the patient, and the judgment of the prescribing physician. Dosage levels of the antibodies for human subjects are generally between about 1 mg per kg and about 100 mg per kg per patient per treatment, such as for example, between about 5 mg per kg and about 50 mg per kg per patient per treatment. In terms of plasma concentrations, the antibody concentrations may be in the range from about 25 Î¼ g/mL to about 500 Î¼ g/mL. However, greater amounts may be required for extreme cases and smaller amounts may be sufficient for milder cases.

Administration of an antibody will generally be performed by a parenteral route, typically via injection such as intra-articular or intravascular injection (e.g., intravenous infusion) or intramuscular injection. Other routes of administration, e.g., oral (p.o.), may be used if desired and practicable for the particular antibody to be administered. An antibody can also be administered in a variety of unit dosage forms and their dosages will also vary with the size, potency, and in vivo half-life of the particular antibody being administered. Doses of a phosphorylation site-specific antibody will also vary depending on the manner of administration, the particular symptoms of the patient being treated, the overall health, condition, size, and age of the patient, and the judgment of the prescribing physician. The frequency of administration may also be adjusted according to various parameters. These include the clinical response, the plasma half-life of the antibody, and the levels of the antibody in a body fluid, such as, blood, plasma, serum, or synovial fluid. To guide adjustment of the frequency of administration, levels of the antibody in the body fluid may be monitored during the course of treatment. Formulations particularly useful for antibody-based therapeutic agents are also described in U.S. Patent App. Publication Nos. 20030202972, 20040091490 and 20050158316. In certain embodiments, the liquid formulations of the application are substantially free of surfactant and/or inorganic salts. In another specific embodiment, the liquid formulations have a pH ranging from about 5.0 to about 7.0. In yet another specific embodiment, the liquid formulations comprise histidine at a concentration ranging from about 1 mM to about 100 mM. In still another specific embodiment, the liquid formulations comprise histidine at a concentration ranging from 1 mM to 100 mM. It is also contemplated that the liquid formulations may further comprise one or more excipients such as a saccharide, an amino acid (e.g., arginine, lysine, and methionine) and a polyol. Additional descriptions and methods of preparing and analyzing liquid formulations can be found, for example, in PCT publications WO 03/106644, WO 04/066957, and WO 04/091658.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the pharmaceutical compositions of the application.

In certain embodiments, formulations of the subject antibodies are pyrogen-free formulations that are substantially free of endotoxins and/or related pyrogenic substances. Endotoxins include toxins that are confined inside microorganisms and are released when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, it is advantageous to remove even low amounts of endotoxins from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with monoclonal antibodies, it is advantageous to remove even trace amounts of endotoxin.

The amount of the formulation that will be therapeutically effective can be determined by standard clinical techniques. In addition, in vitro assays may optionally be used to help identify optimal dosage ranges. The precise dose to be used in the formulation will also depend on the route of administration, and the seriousness of the disease or disorder, and should be decided according to the judgment of the practitioner and each patient's circumstances. Effective doses may be extrapolated from dose-response curves derived from in vitro or animal model test systems. The dosage of the compositions to be administered can be determined by the skilled artisan without undue experimentation in conjunction with standard dose-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula:

Dose (mL)=[patient weight (kg)×dose level (mg/kg)/drug concentration (mg/mL)]

For the purpose of treatment of disease, the appropriate dosage of the compounds (for example, antibodies) will depend on the severity and course of disease, the patient's clinical history and response, the toxicity of the antibodies, and the discretion of the attending physician. The initial candidate dosage may be administered to a patient. The proper dosage and treatment regimen can be established by monitoring the progress of therapy using conventional techniques known to those of skill in the art.

The formulations of the application can be distributed as articles of manufacture comprising packaging material and a pharmaceutical agent which comprises, e.g., the antibody and a pharmaceutically acceptable carrier as appropriate to the mode of administration. The packaging material will include a label that indicates that the formulation is for use in the treatment of prostate cancer.

The following Examples are provided only to further illustrate the invention, and are not intended to limit its scope, except as provided in the claims appended hereto. The present invention encompasses modifications and variations of the methods taught herein which would be obvious to one of ordinary skill in the art.se-response studies. Relevant circumstances to be considered in making those determinations include the condition or conditions to be treated, the choice of composition to be administered, the age, weight, and response of the individual patient, and the severity of the patient's symptoms. For example, the actual patient body weight may be used to calculate the dose of the formulations in milliliters (mL) to be administered. There may be no downward adjustment to “ideal” weight. In such a situation, an appropriate dose may be calculated by the following formula:

Dose (mL)=[patient weight (kg)×dose level (mg/kg)/drug concentration (mg/mL)]

For the purpose of treatment of disease, the appropriate dosage of the compounds (for example, antibodies) will depend on the severity and course of disease, the patient's clinical history and response, the toxicity of the antibodies, and the discretion of the attending physician. The initial candidate dosage may be administered to a patient. The proper dosage and treatment regimen can be established by monitoring the progress of therapy using conventional techniques known to those of skill in the art.

The formulations of the application can be distributed as articles of manufacture comprising packaging material and a pharmaceutical agent which comprises, e.g., the antibody and a pharmaceutically acceptable carrier as appropriate to the mode of administration. The packaging material will include a label that indicates that the formulation is for use in the treatment of prostate cancer.

The following Examples are provided only to further illustrate the invention, and are not intended to limit its scope, except as provided in the claims appended hereto. The present invention encompasses modifications and variations of the methods taught herein which would be obvious to one of ordinary skill in the art.

### VII. Examples

### A. Phosphorylation of mTOR

1. Introduction

The mammalian target of rapamycin (mTOR) Ser/Thr kinase is the catalytic component of two evolutionarily conserved signaling complexes. mTOR signaling complex 1 (mTORC1) is a key regulator of growth factor and nutrient signaling. S6 kinase (S6K) is the best characterized downstream effector of mTORC1. mTOR signaling complex 2 (mTORC2) has a role in regulating the actin cytoskeleton and activating Akt through S473 phosphorylation. Herein we demonstrate that mTOR is phosphorylated differentially when associated with mTORC1 and mTORC2 and that intact complexes are required for these mTORC-specific mTOR phosphorylations. Specifically, we find that mTORC1 contains mTOR phosphorylated predominantly on S2448 whereas mTORC2 contains mTOR phosphorylated predominantly on S2481. Using S2481 phosphorylation as a marker for mTORC2 sensitivity to rapamycin, we find that mTORC2 formation is in fact rapamycin-sensitive in several cancer cell lines in which it had been previously reported that mTORC2 assembly and function were rapamycin-insensitive. Thus phospho-S2481 on mTOR serves as a biomarker for intact mTORC2 and its sensitivity to rapamycin.

In accordance with aspects of the invention provided above, we have demonstrated that mTOR associated with mTORC1 or mTORC2 is phosphorylated on different sites. The rapamycin-sensitive mTORC1 complex contains phospho-S2448, which is consistent with S2448 phosphorylation being sensitive to acute rapamycin treatment. The rapamycin-insensitive mTORC2 complex contains phospho-S2481, which is consistent with S2481 being a rapamycin-insensitive autophosphorylation site. In all the cell lines we tested, the amount of mTOR recovered from Rictor IPs and the amount of S2481 phosphorylation of mTOR were reduced dramatically in response to prolonged rapamycin treatment. We have found a pharmacodynamic biomarker that directly monitors the effects of rapamycin on mTORC2 assembly and function. In several of the cancer cell lines tested the amount of Akt phosphorylated on S473 either increased or remained unchanged, as previously reported (Sarbassov et al., Mol Cell, 22:159-68 (2006)). Thus, S2481 phosphorylation of mTOR is a better marker for the amount of intact mTORC2 in the cell than is phospho-S473 Akt. Because Akt activation is downstream of both PI-3K and mTORC2, using S473 phosphorylation as a readout for mTORC2 does not differentiate between changes in PI-3K activity and changes in mTORC2 activity. Phospho-S2481 serves as a useful biomarker that distinguishes mTOR2 activity from PI-3K activity, which will make it an invaluable tool when evaluating inhibitors that are specific for mTORC2 only.

It has been reported that S2481 is a rapamycin-insensitive autophosphorylation site (Peterson et al., J Biol Chem, 275:7416-23 (2000)). We have shown that phosphorylation of S2481 is dependent on intact mTORC2 and that this site is sensitive to prolonged rapamycin treatment. The sensitivity to rapamycin is most likely due to prolonged treatment inhibiting the assembly of mTORC2 (Sarbassov et al., Mol Cell, 22:159-68 (2006)). Rictor and/or mSin1 may hold mTOR in a conformation that is amenable to autophosphorylation, perhaps in trans within an mTOR dimer. We are currently exploring why S2481 phosphorylation requires intact mTORC2.

The fact that mTOR phosphorylation at S2448 and S2481 sites are highly conserved across vertebrate species points to phosphorylation having a role in mTOR regulation (FIG. 1A). Alignment of multiple mTOR orthologs reveals that, while present in all vertebrate species analyzed, both phosphorylation sites analyzed here are absent in invertebrates (FIG. 1A). In fact, the entire region between the kinase and FATC domains is extremely well-conserved throughout vertebrate species but highly variable in other species, even closely related members of the same genus (FIG. 1A). The deletion of amino acids 2430-2450 within this region leads to an elevated level of mTOR kinase activity (Sekulic et al., Cancer Res, 60:3504-13 (2000)). In the AGC family of protein kinases, the C-terminal tail has evolved as a regulatory module that is necessary for catalytic activity through various interactions with the catalytic domain (Kannan et al., Proc Natl Acad Sci USA, 104:1272-7 (2007)). The C-terminal region of mTOR could modulate catalytic activity in a similar fashion.

Our data demonstrate the existence and the identity of mTORC-specific phosphorylation sites on mTOR, and that phospho-S2481 can be used as a specific marker to detect intact mTORC2 within the cell. Because S2448 and S2481 have evolved recently in vertebrate mTOR, they may regulate TOR activity in a manner not found in invertebrates.

2. Materials and Methods

Antibodies and reagents. The following antibodies were purchased from Cell Signaling Technology: phospho-mTOR(S2448 and S2481), Akt and phospho-Akt (S473) rabbit polyclonal antibodies. Phospho-S6K (T389) and mTOR (mTab1) rabbit polyclonal antibodies were purchased from Millipore. Rictor and mSin1 rabbit polyclonal antibodies were purchased from Bethyl Laboratories. The anti-Raptor rabbit antiserum was developed with the antibody service from Invitrogen utilizing the peptide PHSHQFPRTRKMFDKG, amino acid sequence 918-933 of human Raptor. Rapamycin was purchased from Sigma. Insulin and IGF-1 were purchased from Research Diagnostics, Inc.

Lentivirus-mediated gene knockdown. We obtained pLKO.1 based short-hairpin constructs specific for mTOR (Addgene plasmid #1855), Raptor (Addgene plasmid #1857), Rictor (Addgene plasmid number #1853), and mSin1 (Addgene plasmid #13483), as well as a scrambled control sequence (Addgene plasmid #1864) from the plasmid repository at Addgene. They have been described previously (Frias et al., Curr Biol, 16:1865-70 (2006); Sarbassov et al., Science, 307:1098-101 (2005)).

Plasmids were co-transfected together with the lentiviral packaging (pMDL), envelope (CMV-VSVG) and rev-expressing (RSV-REV) constructs into actively growing HEK293T cells using the Effectene transfection reagent (Qiagen) per manufacturer's protocol. Virus-containing supernatants were collected 48 hr. after transfection. Cells were infected twice in the presence of 1 μg/ml polybrene, selected for puromycin resistance and analyzed 48-72 hr. post-infection.

Cell culture, cell lysis and immunoprecipitation. All cells were cultured in DMEM/10% FCS supplemented with penicillin, streptomycin and ciprofloxacin at 37° C. Where applicable, cells were cultured in serum-free DMEM for 24 hr. prior to growth factor stimulation.

Cells were rinsed 2× with cold PBS and lysed in 1 ml 50 mM Hepes pH 7.5, 150 mM NaCl, 10% glycerol, 0.3% Chaps (v/v), 1.5 mM MgCl₂, 1 mM EGTA, 100 mM NaF, 500 μM sodium orthovanadate, 10 μg/ml aprotinin, and 10 μg/ml leupeptin per 10 cm tissue culture dish. Lysates were rotated end over end at 4° C. for 20 min. and clarified by centrifugation at 12,000 rpm for 10 min at 4° C. Protein concentrations were determined using the Bio-Rad DC Protein Assay kit according to manufacturer's protocol. Lysates were either mixed v/v with 2×SDS sample buffer or subjected to immunoprecipitation.

For immunoprecipitation, lysates were incubated with the appropriate antibody while being mixed end over end for 1 hr at 4° C. Protein A-Sepharose was added and the samples were mixed for an additional hr. at 4° C. Immune complexes were isolated by centrifugation and washed 4× with ice-cold lysis buffer. Samples were boiled for 5 min. at 100° C. in lx SDS sample buffer.

Immunoblot analysis. Immunoprecipitates or whole-cell lysates normalized for total protein concentration were resolved by SDS-PAGE and proteins were electrotransferred to PVDF membranes. Immunoblotting was performed per manufacturer's protocol, and reactive proteins were visualized by ECL.

3. Results

mTOR has several phosphorylation sites in the 60 amino acid region beyond the catalytic domain in the C-terminal tail, and these sites are conserved in all vertebrates but not in invertebrates (FIG. 1A). In fact, the entire 60 residue region containing these sites is highly conserved among vertebrate species, suggesting it could be a vertebrate-specific regulatory element (FIG. 1A). Because the regulation of mTORC1 and mTORC2 formation is poorly understood, we set out to analyze whether mTOR phosphorylation has any effect on either complex formation. Rictor and Raptor immunoprecipitates (IPs) from untreated serum-starved HEK293 cells and cells treated with 200 nM insulin for 5 min. were analyzed by immunoblotting with antibodies specific for either total mTOR, mTOR phosphorylated on S2448 or mTOR phosphorylated on S2481. Whole cell lysates were analyzed as controls to insure that insulin stimulation led to increased S2448 and S2481 phosphorylation. mTOR phosphorylated on S2448 was mainly associated with Raptor, whereas mTOR phosphorylated on S2481 was predominantly associated with Rictor in HEK293 cells (FIG. 1B). The amount of mTOR associated with either Raptor or Rictor did not change as a result of insulin stimulation and concomitant mTOR phosphorylation. Rictor and Raptor IPs from actively growing U2OS cells were also analyzed to confirm that this result was not specific to HEK293 cells. As in HEK293 cells, mTOR phosphorylated on S2448 was associated with Raptor and mTOR phosphorylated on S2481 was associated with Rictor in U2OS cells (FIG. 1C). However, there was a low level of S2448 phosphorylation associated with mTORC2 in HEK293 cells that was not observed in actively growing U2OS cells (FIGS. 1B and C

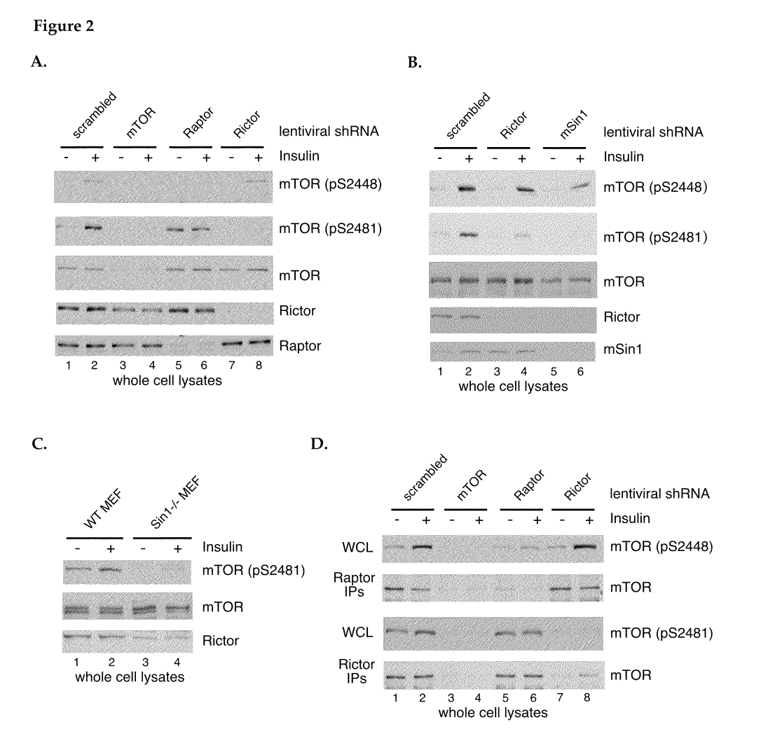

While our data demonstrated that S2448 phosphorylation of mTOR is associated with mTORC1 and that S2481 phosphorylation of mTOR is associated with mTORC2, it was unclear if intact mTORC1 and mTORC2 complexes are required for these mTOR phosphorylations. To investigate this, short hairpin RNA (shRNA) sequences that specifically deplete endogenous mTOR, Rictor or Raptor were expressed in HEK293 cells via lentiviral infection. Three days after infection, cells were serum-starved overnight, control and insulin-stimulated cells were lysed, and whole cell lysates were analyzed by immunoblotting with antibodies that recognize either phospho-S2448 or phospho-S2481. In cells in which Raptor levels were significantly reduced, we found complete ablation of insulin-stimulated S2448 phosphorylation without any effect on S2481 phosphorylation. Conversely, in cells in which Rictor had been depleted, insulin-stimulated S2481 phosphorylation was abolished without any reduction in S2448 phosphorylation. The data demonstrate that intact mTORC1 is necessary for S2448 phosphorylation and that intact mTORC2 is necessary for S2481 phosphorylation, further underscoring the specificity of these mTOR phosphorylation sites for the different mTOR signaling complexes (FIG. 2A). Although the depletion of mTOR, Raptor and Rictor was not as efficient in U2OS cells as in HEK293 cells, decreased Raptor expression led to diminished S2448 phosphorylation, and decreased Rictor expression led to diminished S2481 phosphorylation in actively growing U2OS cells (data not shown). To confirm that mTORC2 is necessary for S2481 phosphorylation we utilized shRNA knockdown of the other major mTORC2 specific component, mSin1 in HEK293 cells. We found that depletion of mSin1 also reduced the level of Rictor, as previously reported (Frias et al., Curr Biol, 16:1865-70 (2006)) (FIG. 2B). In cells with reduced levels of both mSin1 and Rictor, the insulin-induced phosphorylation of mTOR on S2481 was completely abolished, confirming that intact mTORC2 is necessary for S2481 phosphorylation (FIG. 2B). To definitively prove that S2481 phosphorylation requires intact mTORC2, we analyzed mTOR phosphorylation in Sin1^{-/-} mouse embryo fibroblasts (MEFs) (Jacinto et al., Cell, 127:125-37 (2006)). Consistent with the shRNA results, the basal and growth factor-induced phosphorylation of S2481 was severely diminished in Sin1^{-/-} MEFs as compared to WT (FIG. 2C). Genetic ablation of Sin1 appears to have less of an effect on S2481 phosphorylation than does shRNA-mediated knockdown of mSin1 (FIGS. 2B and C). This is most likely due to the presence of a low level of Rictor in Sin1^{-/-} MEFs [(Jacinto et al., Cell, 127:125-37 (2006)) and FIG. 2C].

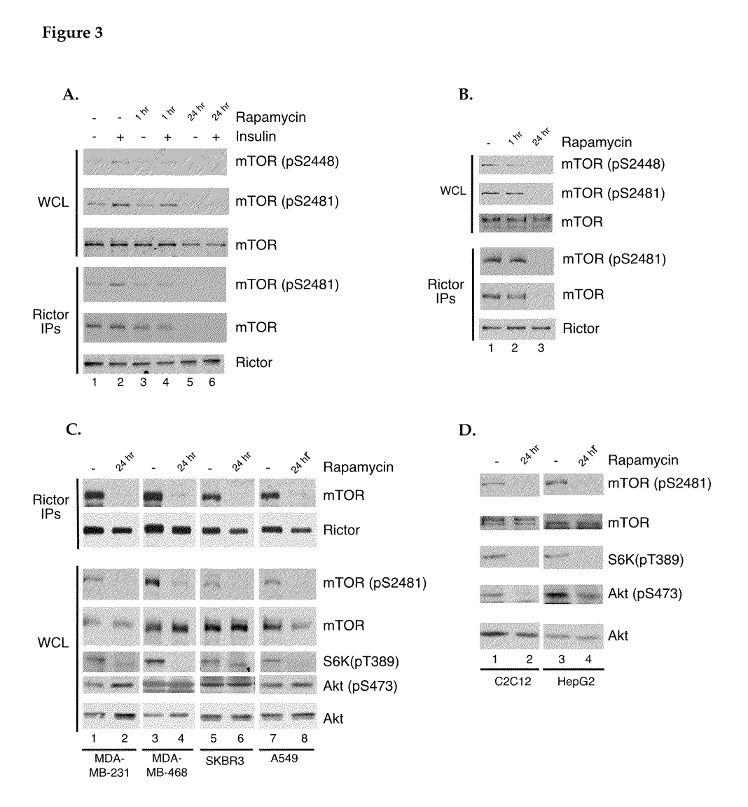

Although insulin-stimulated S2481 phosphorylation remained unchanged in Raptor-depleted cells, basal levels of S2481 phosphorylation were higher (FIG. 2A). Careful examination of the data from several independent experiments show that in some cases S2481 phosphorylation was less effectively abolished by serum depletion than S2448 phosphorylation, and this was independent of Raptor knockdown (for example, compare the insulin-induced S2481 phosphorylation shown in FIGS. 2A, 2B, 2D and 3A). However, others have reported findings that indirectly suggest that Raptor knockdown may have an effect on mTORC2, possibly as a result of freeing up more mTOR to interact with Rictor (Yang et al., Genes Dev, 20:2820-32 (2006); Sarbassov et al., Science, 307:1098-101 (2005)). To test this, we performed Rictor IPs from cells in which Raptor was depleted and Raptor IPs from cells in which Rictor was depleted and compared the level of mTOR associated with each protein. Although there was a significant decrease in the level of S2448 phosphorylation, which indicates efficient Raptor depletion, there was no discernible change in the amount of mTOR associated with Rictor in cells with decreased Raptor expression (FIG. 2D). In Rictor-depleted cells, S2481 phosphorylation was completely abolished, yet the levels of mTOR associated with Raptor were unchanged (FIG. 2D). These data provide direct evidence that the relative amount of intact mTORC1 has no effect on the relative amounts of intact mTORC2 and vice versa.

The phosphorylation of mTOR on S2481 and the assembly and function of mTORC2 were initially reported to be rapamycin-insensitive (Sarbassov et al., Curr Biol, 14:1296-302 (2004); Peterson et al., J Biol Chem, 275:7416-23 (2000)), but more recent studies indicate that prolonged rapamycin treatment inhibits both mTORC2 assembly and function (Sarbassov et al., Mol Cell, 22:159-68 (2006)). Because S2481 phosphorylation requires intact mTORC2, we tested whether prolonged rapamycin treatment had any effect on S2481 phosphorylation. Whole cell lysates from control and insulin-stimulated cells treated with 100 nM rapamycin for either one or 24 hr were analyzed for phosphorylation of mTOR on S2448 and S2481. We observed a marked reduction of S2448 phosphorylation in insulin-stimulated cells with either acute or prolonged treatment of rapamycin (FIG. 3A). In contrast, insulin-stimulated S2481 phosphorylation showed no discernible decrease after acute treatment with rapamycin but was completely absent after prolonged treatment (FIG. 3A). When Rictor IPs from these same cells were analyzed for bound mTOR, we observed a slight decrease in the amount of mTOR bound to Rictor after one hr of rapamycin treatment, indicating that acute rapamycin treatment may have a minor, yet reproducible, effect on mTORC2 formation. However, after 24 hr treatment, no detectible mTOR was bound to Rictor, indicating that prolonged rapamycin treatment inhibits the assembly of mTORC2 in HEK293 cells (FIG. 3A). In addition, both mTORC2 assembly and S2481 phosphorylation were inhibited by 24 hr but not one hr rapamycin treatment in actively growing U2OS cells (FIG. 3B).

Intriguingly, U2OS cells were reported to be insensitive to prolonged rapamycin treatment when phosphorylation of Akt at S473 was utilized as a marker for mTORC2 function (Sarbassov et al., Mol Cell, 22:159-68 (2006)). This led us to analyze S2481 phosphorylation and mTORC2 assembly in response to prolonged rapamycin treatment in several other cancer cell lines in which S473 phosphorylation is reported to be insensitive to rapamycin (Sarbassov et al., Mol Cell, 22:159-68 (2006)). Analysis of mTOR S2481 and Akt S473 phosphorylation in whole cell lysates of MDA-MB-231, MDA-MB-468, SKBR3 and A549 cells treated with 100 nM rapamycin for 24 hr showed that mTOR S2481 phosphorylation was greatly diminished (FIG. 3C) while Akt phosphorylation remained unchanged or increased, as reported [(Sarbassov et al., Mol Cell, 22:159-68 (2006)) and FIG. 3C]. However, when Rictor IPs were analyzed for bound mTOR in parallel, the amount of mTOR was dramatically reduced, if not completely abolished (FIG. 3C). The decreased amount of mTOR bound to Rictor paralleled the reduction in S2481 phosphorylation. As a control, we analyzed the phosphorylation of mTOR on S2481 and Akt on S473 in C2C12 myoblasts and HepG2 cells, two cell lines that were reported to be sensitive to prolonged rapamycin treatment (Sarbassov et al., Mol Cell, 22:159-68 (2006)). As expected, both S2481 and S473 phosphorylation were sensitive to rapamycin treatment in these cells (FIG. 3D). Our data demonstrate that phosphorylation of S2481 on mTOR is a more direct marker of intact mTORC2 than is phosphorylation of S473 of Akt. We assert that mTOR S2481 phosphorylation is a biomarker that can be used to analyze the sensitivity of mTORC2 to rapamycin treatment in various cancer types.

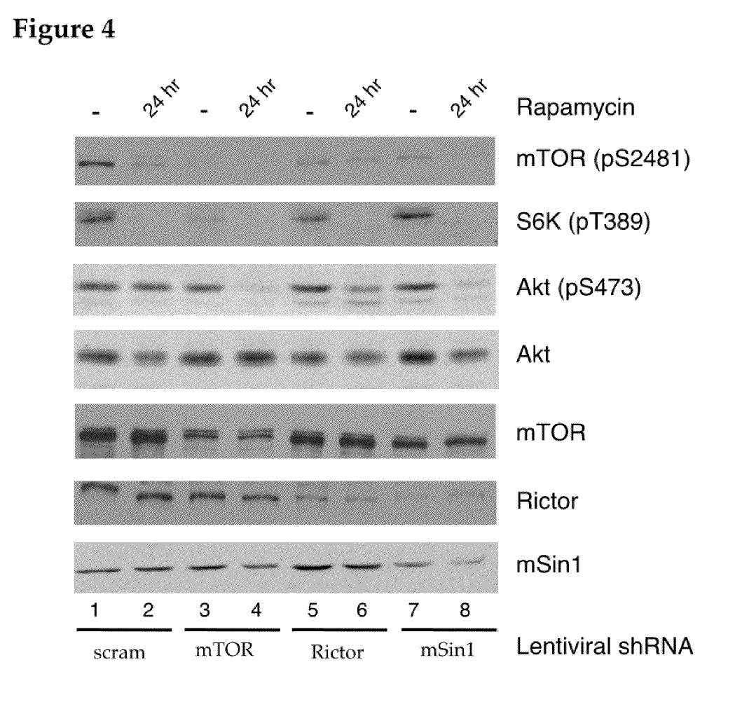

Cells with rapamycin-insensitive Akt phosphorylation are reported to become sensitive to rapamycin treatment after partially reducing mTOR expression (Sarbassov et al., Mol Cell, 22:159-68 (2006)). One explanation is that even a small amount of intact mTORC2 can sustain robust Akt S473 phosphorylation in these cells, and that prolonged rapamycin treatment is not enough to decrease mTORC2 levels below the threshold necessary for Akt phosphorylation. Another possibility is that in certain cancer settings mTOR can phosphorylate Akt independently of its association with either Rictor or mSin1. To test this, we analyzed whether partial knockdown of either Rictor or mSin1 could render cells sensitive to rapamycin treatment. mTOR, Rictor or mSin1 expression was reduced by shRNA expression in MDA-MB-468 cells. Following rapamycin treatment for 24 hr, whole cell lysates were analyzed for mTOR S2481 phosphorylation and Akt S473 phosphorylation. As expected, a decrease in mTORC2, either by shRNA, rapamycin treatment, or both, led to a reduction in the amount of mTOR phosphorylated on S2481 (FIG. 4). Partial depletion of Rictor led to a mild, yet reproducible decrease in S473 phosphorylation upon treatment with rapamycin (FIG. 4). Partial depletion of mSin1 had a much more profound effect on S473 phosphorylation upon prolonged rapamycin treatment (FIG. 4). This is most likely due to a decrease in mSin1 protein levels leading to a concomitant decrease in Rictor protein levels, making mSin1 knockdown a more efficient way to diminish mTORC2 levels in the cell. Partial depletion of mTOR had the greatest effect on S473 phosphorylation upon prolonged rapamycin treatment. This makes sense, as mTOR is the catalytic component of the mTORC2 complex. These results indicate that rapamycin treatment alone is not enough to completely disrupt mTORC2 formation below levels that are necessary for S473 phosphorylation and that mTOR still requires Rictor/mSin1 in these cells to mediate S473 phosphorylation.

### B. Breast Cancer Studies

1. Introduction

As the second leading cause of cancer death in women, breast cancer leads to 350,000 deaths per year worldwide. Recent studies have advanced the understanding of the signaling pathways involved in breast cancer onset, leading to the development of several novel targeted therapies. The Ser/Thr kinase mammalian target of rapamycin (mTOR) has emerged as a key target of cancer therapeutics, as it regulates many oncogenic pathways. Currently, the allosteric mTOR inhibitor rapamycin and its “rapalog” derivatives are in several promising clinical trials, and a new subset of molecules that directly inhibit the mTOR kinase domain are being evaluated as potential oncogenic therapies. There is evidence suggesting anticancer activity when temsirolimus and another rapalog, everolimus are used in conjunction with endocrine therapy in breast cancer. This is most likely due to crosstalk between hormone receptors and mTOR signaling. mTOR regulation is extremely complex, and a robust biomarker that is a direct readout for target inhibition has not been identified. Current strategies to analyze mTOR in the presence of rapamycin include using the phosphorylation of a downstream target, Akt, as a marker for activity. However, Akt phosphorylation is often unchanged in the presence of rapamycin while there are clear perturbations to mTOR activity.

As discussed above, we have identified the first biomarker that is a direct readout for mTOR activity, namely phospho-S2481, which is an mTOR autophosphorylation site. Phosphorylation of S2481 is specific for mTORC2 and can be used as a marker to determine the rapamycin sensitivity of mTORC2 formation in several cancer cell lines that were reported to be insensitive to prolonged rapamycin treatment as deduced using the downstream phosphorylation of S473 of Akt as a marker. As demonstrated herein, apamycin suppresses the formation of mTORC2 in all previously described “rapamycin-insensitive” cancer cell lines tested, and the lack of S2481 phosphorylation correlates with mTORC2 dissolution.

Aberrant activation of the PI3K-Akt pathway contributes to many human cancers, including breast cancer. The mechanism of such oncogenic activation is usually either hyper-activated receptor tyrosine kinases (RTKs) upstream of PI3K or genetic alterations of specific components of the pathway including PTEN deletion and activating mutations of PI3K and Akt. HER2, a member of the epidermal growth factor (EGF) RTK family, is overexpressed in 25% of human breast cancer cases and confers more aggressive tumors and poor prognosis. HER2 receptor activation is directly upstream of several survival pathways, including PI3K-Akt. Anti-HER2 therapies, such as Herceptin, can markedly improve survival when combined with chemotherapy in patients in metastatic breast cancers that overexpress HER2. However, mutations in effectors downstream of HER2 can confer resistance to anti-HER2 therapeutics. For example, the loss of PTEN in HER2 over-expressing breast cancers predicts Herceptin resistance because PTEN activity is necessary for tumor inhibition by Herceptin. Hyper-activation of HER2 and PI3K, as well as the loss of PTEN function, all lead to dysregulation of Akt. Because mTORC2 phosphorylation at the HM is necessary for maximal Akt activation, mTOR is a key regulator of one of the most frequently altered signaling pathways in breast cancer.

Finally, approximately 70% of invasive breast cancers are positive for estrogen receptor (ER) and progesterone receptor (PR) expression at the time of diagnosis. There is evidence that signaling downstream of ER and PR and the pathways regulated by RTKs are intertwined. PI3K can phosphorylate and activate ER, but the interaction between PI3K and ER can also serve to localize PI3K to the cell membrane where it can activate Akt. Growth factor-induced activation of PI3K-Akt signaling reduces the levels of PR mRNA levels and low PR levels can indicate high HER2 activity. Therefore, inhibition of mTOR is a therapeutic option for breast cancer. However, only 10% of unselected breast cancer patients responded to treatment with the rapamycin analog temsirolimus, yet a separate randomized trial utilizing endocrine therapy plus evirolimus demonstrated that everolimus significantly increases the efficacy of endocrine therapy in ER positive breast cancer patients (Raymond, 2004; Baselga, 2009).

2. Results

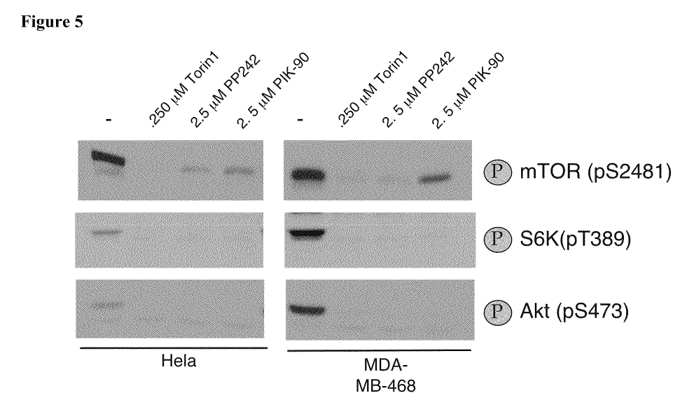

To test whether the phosphorylation of S2481 can be used to monitor mTOR inhibition in response to mTKIs, we obtained Torinl from David Sabatini and PP242 from Kevan Shokat, and we treated actively growing Hela and MDA-MB-468 cells with these inhibitors at the indicated concentrations for 1 h at 37° C. The PI3K specific inhibitor PIK-90 was also obtained from the Shokat lab for use as a control. Whole cell lysates were then analyzed for mTOR phosphorylation at S2481. Phosphorylation of the mTORC1 substrate S6K at T389 and the mTORC2 substrate Akt at S473 were analyzed as controls. Both Torin1 and PP242 inhibited the phosphorylation of substrates downstream of both mTOR signaling complexes. Both inhibitors abolished mTOR autophosphorylation at S2481. See FIG. 5. Therefore, S2481 phosphorylation is a marker for mTORC2 inhibition in response to mTKIs as well as chronic rapamycin treatment. Thus, S2481 is a biomarker for mTORC2 activity in the cell. Intriguingly, our data show that treatment of cells with the PI3K inhibitor PIK-90 also leads to a reduction in S2481 phosphorylation.

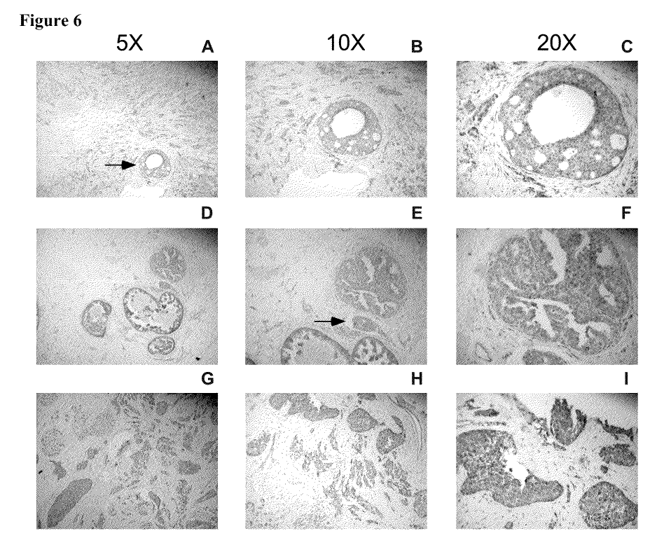

We also tested a rabbit polyclonal phospho-specific S2481 antibody commercially available from Millipore in immunohistochemistry (IHC). We stained paraffin-embedded histological sections from breast tumor tissue derived from human patients suffering from invasive ductal carcinoma. These samples were derived from frozen tissue blocks and obtained from the University of CA, San Diego Department of Pharmacology in collaboration with Dr. Michael Peterson. The first tumor is grade 1 with pathological staging pT1cN0MX and is stage I breast cancer. See FIG. 6A-C. The second tumor is grade 3 with pathological staging pT2N1MX and is stage IIb breast cancer. See FIG. 6D-F. The third tumor is grade 3 with pathological staging pT2N2aMX and is stage IIIa breast cancer. These sections were counterstained with hematoxylin. Our data show that the phospho-specific S2481 antibody from Millipore works in IHC. See FIG. 6A-I. In all three cases, we see an increase in phospho-S2481 in the tumor tissue over the normal breast tissue. See FIG. 6A-I.

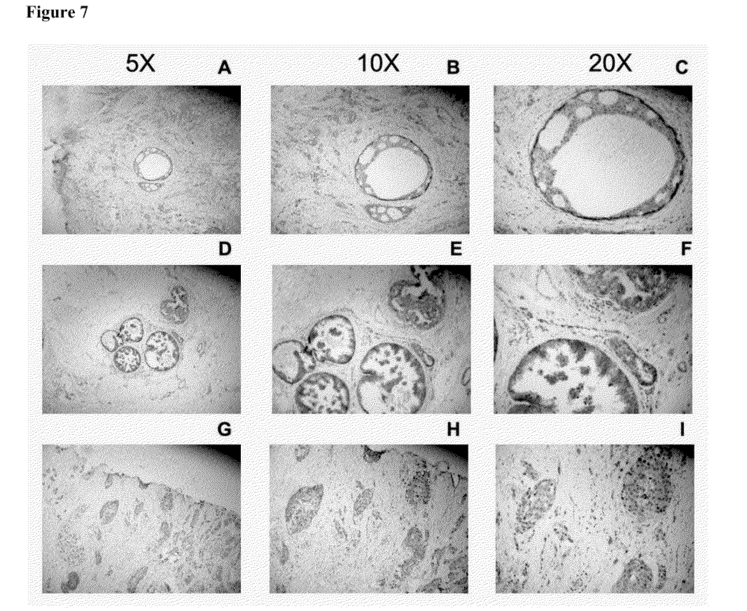

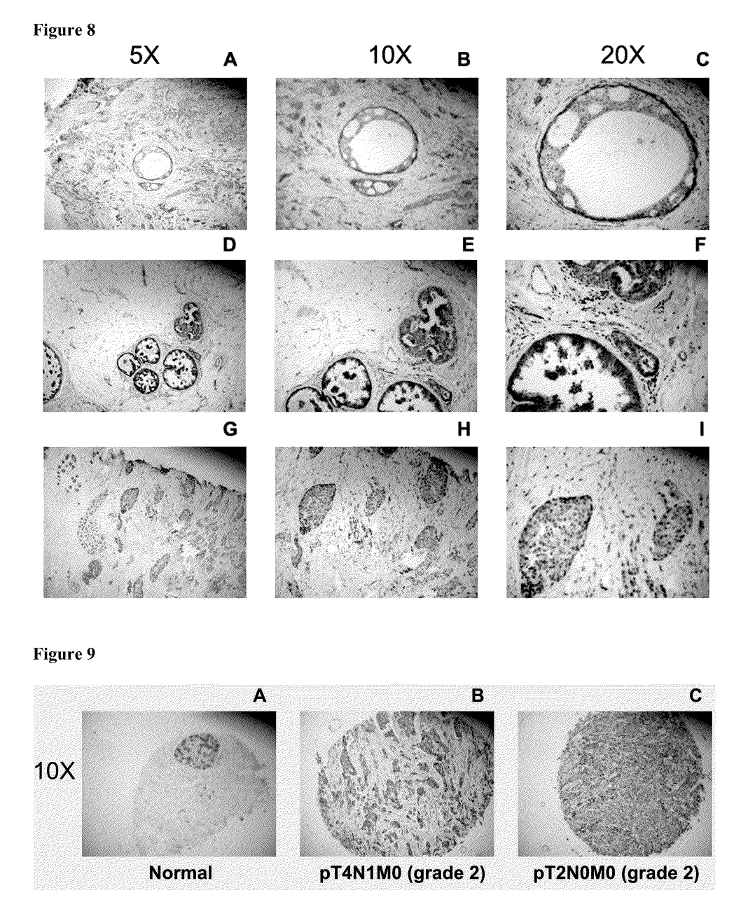

We have stained histological sections derived from the same frozen tumor tissue used in the procedures described for FIG. 6A-I for Akt phosphorylated on S473 downstream of mTORC2 (FIGS. 7A-I) and for T389 downstream of mTORC1 (FIG. 8A-I). These antibodies are available from Cell Signaling Technology (CST) and Millipore, respectively. Our data show that there are elevated levels of both phospho-Akt and phospho-S6K in and around the same areas that have elevated levels of phospho-S2481. Therefore, the phosphorylation of S2481 can be used as a biomarker to detect elevated mTOR activity in tumor tissue.

We purchased a high-density breast invasive ductal and lobular carcinoma tissue array from Biomax, Inc. This array contains 80 cases of invasive ductal carcinoma, 80 cases of invasive lobular carcinoma and 32 examples of normal or normal adjacent breast tissue. We stained this array with the phospho-S2481 antibody. Representative staining of a normal breast tissue control (FIG. 9A), a case of stage Mb invasive ductal carcinoma (FIG. 9B), and a case of stage I invasive ductal carcinoma (FIG. 9C) are shown. These examples clearly demonstrate that there is more phospho-S2481 staining in the tumor tissue from more advanced stages of breast cancer. Compare FIG. 9B to 9C.

3. Additional Breast Cancer Studies

a) Additional Breast Cancer Study 1

Patient samples will be obtained as tumor tissues arrays from Biomax, Inc. The company has multiple samples available with information on clinical stage and pathological grade. This will allow us to analyze the amount of S2481 phosphorylation at any given disease state. We will also analyze samples from normal, non-cancerous breast tissue as a control. Samples will be stained with the Millipore phospho-S2481 antibody at 1:100 dilution and detected using Vector Laboratories Vectastain ABC detection kit per the manufacturer's protocol. This is the same method used to detect S2481 phosphorylation via IHC in our preliminary experiments. Tissues will also be stained for the presence of Akt phosphorylated on S473 as a control for mTORC2 activity. The rabbit monoclonal antibody D9E from CST recognizes Akt specifically phosphorylated on S473 in IHC. We have utilized this antibody at a 1:50 dilution per the manufacturer's protocol. See FIG. 7A-I. To analyze mTORC1 activity, we will stain for the presence of phospho-S6K. We have utilized a polycolonal antibody from Millipore to stain for S6K phosphorylated on T389. This antibody works at a 1:250 dilution. See FIG. 8A-I. We will control for the levels of mTOR in these samples by staining with an antibody that recognizes total mTOR. This antibody is available from Millipore and is certified to work in IHC.

We will score the stage of the breast cancer progression against the amount of S2481 phosphorylation detected in an effort to correlate mTOR activity with tumor progression. We will score the intensity of staining on a semi-quantitative basis. If the amount of phosphorylation is increased in later stages of more aggressive tumors then it is possible that S2481 phosphorylation can be used as a diagnostic biomarker.

Phospho-S2481 levels may also demonstrate the amount of signaling through the mTORC2 pathway that is occurring in a particular tumor, which may correlate with the response to inhibiting this pathway.

b) Additional Breast Cancer Study 2

We will analyze the same breast cancer tumor tissue arrays described in above for HER2 and PTEN protein levels. There are antibodies against both HER2 and PTEN commercially available from CST. We will also stain for the presence of these hormone receptors because of the crosstalk between PI3K-Akt, mTOR and endocrine signaling. ER and PR antibodies that work in IHC are commercially available from Millipore and CST, respectively.

There are breast tumor tissue arrays available from Biomax that contain the information on the HER2/ER/PR status of the patient. We stain these arrays for the presence of mTOR that is phosphorylated on S2481. We will then score the amount of S2481 phosphorylation and determine the correlation between the expression levels of HER2, ER and PR. We will also stain these arrays for the presence of PTEN to determine the effect of loss of PTEN on phospho-S2481 levels. We will stain the available breast tumor tissue arrays (including the array used in FIG. 7) for HER2, PTEN, ER and PR.

c) Additional Breast Cancer Study 3

We will utilize lentiviral short hairpin (sh)RNA to knockdown the expression of PI3K to determine its role in signaling to mTORC2. PI3K is a multi-subunit enzyme with each subunit having several isoforms. Two of the four isoforms of the catalytic subunit, p 110α and p110β, are ubiquitously expressed and are involved in insulin receptor signaling. We will begin by depleting p110α and p110β both separately and together, in actively growing HEK 293 cells and we will analyze whole cell lysates for the presence of mTOR phosphorylated on S2481. A decrease in S2481 levels when we decrease the expression of p110α and/or p110β will suggest that PI3K is necessary for mTORC2 activation. We will also utilize a constitutively active form of the p110α catalytic subunit that is targeted to the cell membrane by myristic acid at its amino-terminus. This construct, along with a control construct that expresses a kinase-dead (KD) mutant, will be expressed in actively growing HEK 293 cells.

### C. Lung Cancer Studies

1. Introduction

The LKB1 Ser/Thr kinase is a tumor suppressor that regulates cell polarity and differentiation, and it responds to cellular energy status in order to regulate metabolism. It is mutated in the autosomal-dominant Peutz-Jeghers syndrome (PJS), leading to hamartomas in the gastrointestinal tract, and it is frequently altered in lung cancer. When cellular energy sources are low, levels of AMP rise. AMP binds to the AMP-activated kinase (AMPK), priming it for phosphorylation and activation by LKB1. Active AMPK regulates mTORC1 in at least two different ways. In the first, AMPK acts directly on mTORC1 by phosphorylating Raptor and reducing mTORC1 activity. In the second manner of regulation, AMPK acts indirectly on mTORC1 by phosphorylating and activating the tuberous sclerosis complex 2 (TSC2) protein. TSC2, along with its obligate binding partner TSC1, is an upstream negative regulator of mTORC1. Intriguingly, loss-of-function mutations in the genes encoding TSC1 and TSC2 lead to tuberous sclerosis, a syndrome, like PJS, that is characterized by the development of hamartomas with a predisposition to malignancy. Loss of heterozygosity (LOH) of both the TSC1 and TSC2 gene loci occurs frequently in both lung adenocarcinomas and pre-invasive lung lesions.

The link between LKB1, mTOR and lung cancer has broad implications for therapy. Studies with temsirolimus and everolimus, two rapamycin analogs, have shown promise in phase II clinical trials for non-small cell lung cancer.

2. Results

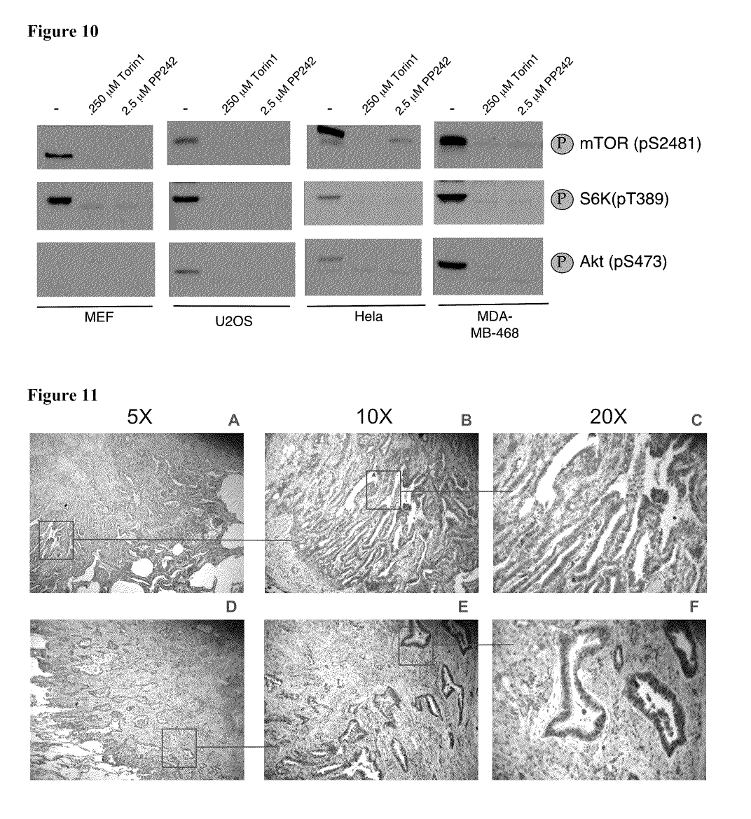

We obtained PP242 from Kevin Shokat and Torin1 from David Sabatini, and we treated actively growing mouse embryonic fibroblasts (MEFs), U2OS, Hela and MDA-MB-468 cells with these inhibitors at the indicated concentrations for 1 h at 37° C. Whole cell lysates were then analyzed for mTOR phosphorylation at S2481. Phosphorylation of the mTORC1 substrate S6K at T389 and the mTORC2 substrate Akt at S473 were analyzed as controls. Both Torin1 and PP242 inhibited the phosphorylation of substrates downstream of both mTOR signaling complexes. See FIG. 10. Both inhibitors abolished mTOR autophosphorylation at S2481. See FIG. 10. Thus, S2481 may is a biomarker for mTORC2 activity in the cell.

We tested a rabbit polyclonal phospho-specific S2481 antibody commercially available from Millipore in immunohistochemistry (IHC). We stained paraffin-embedded histological sections from lung tumors derived from human patients suffering from invasive lung adenocarcinoma. These samples were derived from frozen tissue blocks and obtained from the University of CA, San Diego Department of Pharmacology in collaboration with Dr. Michael Peterson. The first tumor is moderately differentiated with pathological staging pT1N0MX (FIG. 11A-C) while the second tumor is moderately to poorly differentiated with pathological staging pT2N0MX (FIG. 11D-F). These sections were counterstained with hematoxylin. Our preliminary data show that the phospho-specific S2481 antibody from Millipore works in IHC. See FIG. 11A-F. In two different cases, we see an increase in phospho-S2481 in the invasive tumor tissue over the normal lung tissue. See FIG. 11A-F.

3. Additional Lung Cancer Studies

a) Additional Lung Cancer Study 1

Patient samples will be obtained from the University of CA, San Diego Department of Pharmacology. We will obtain samples that are in various stages of tumor progression so that we may analyze the amount of S2481 phosphorylation at a given disease state. We will also analyze samples from normal, non-cancerous lung tissue as a control. To maximize the number of samples available to us, lung tumor tissue arrays will be acquired from Biomax, Inc. for staining, as well. Several arrays are available with information on clinical stage and pathological grade. Samples will be stained with the Millipore phospho-S2481 antibody at 1:100 dilution and detected using Vector Laboratories Vectastain ABC detection kit per the manufacturer's protocol. This is the same method used to detect S2481 phosphorylation via IHC in experiments outlined above. Tissues will also be stained for the presence of Akt phosphorylated on S473 as a control for mTORC2 activity. Phospho-specific Akt antibodies that have been validated in IHC are available from Cell Signaling Technologies (CST). To analyze mTORC1 activity, we will stain for the presence of phospho-S6K and phospho-4E-BP1. We will also control for the levels of mTOR in these samples by staining with an antibody that recognizes total mTOR. Antibodies against these proteins are commercially available from CST. We will score the stage of the lung tumor progression against the amount of S2481 phosphorylation detected in an effort to correlate mTORC2 activity with tumor progression.

b) Additional Lung Cancer Study 2

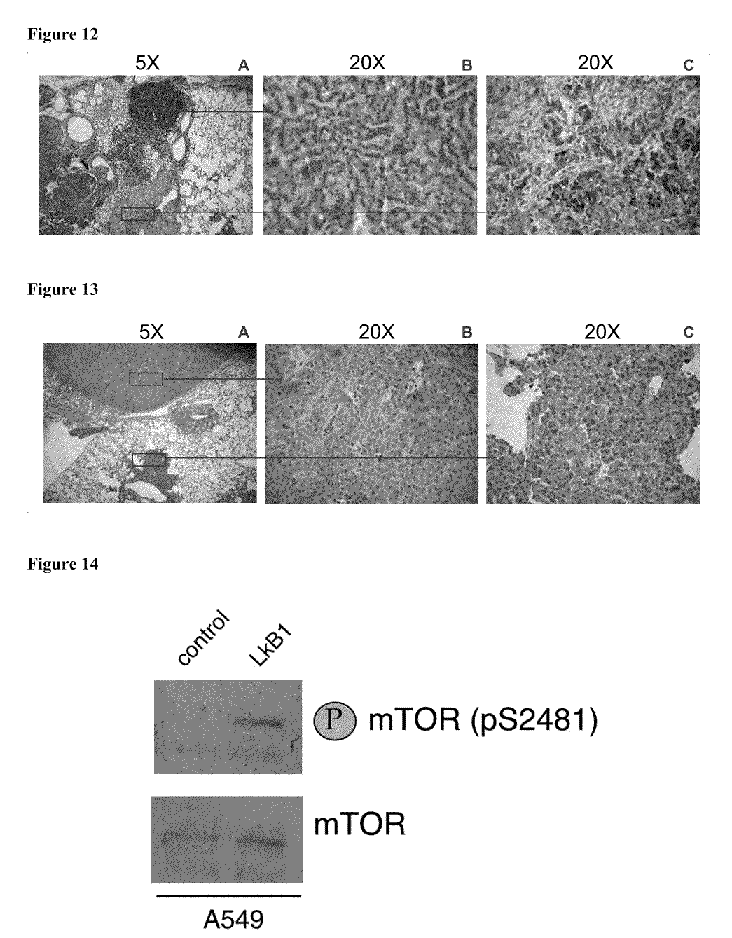

While testing the phospho-S2481 for use in IHC, we stained paraffin-embedded histological sections from lung tumors derived from mice containing a conditional allele of a gain-in-function oncogenic K-ras mutant crossed with mice that are conditionally heterozygous for the LKB1 gene (referred to as K-ras X LKB+/−). See FIG. 12A-C. The sections were counterstained with hematoxylin. In these mouse sections, there is staining for mTOR phosphorylated on S2481 in both the nucleus and the cytoplasm. See FIGS. 12B and 12C. In the human sections, only the cytoplasm is positive for S2481 phosphorylation. However, we have immunofluorescence data that shows phospho-S2481 present in the nucleus in tissue culture cells derived from mice. Intriguingly, our data indicate that in areas of more advanced stages of tumorigenesis, there is less mTOR phosphorylated on S2481. See FIGS. 12B and 12C. The tissue stained in FIG. 12B consists of a uniform population of epithelial cells and resembles an adenoma, while the pattern in FIG. 12C has more cytological atypia and regional variation, which is more indicative of adenocarcinoma.

We stained tissue sections of lung tumors derived from mice that are conditionally homozygous deficient for the LKB1 gene crossed with mice carrying the conditional gain-in-function oncogenic K-ras mutant allele (referred to as K-ras X LKB−/−). There is a significant decrease in phosphorylation of mTOR on S2481 on lung tumors derived from LKB1 deficient mice. See FIG. 13A-C.

We analyzed S2481 phosphorylation in whole cell lysates derived from A549 cells that were reconstituted with an retroviral expression construct for the wild-type (WT) LKB1 protein and compared it to S2481 phosphorylation from A549 cells reconstituted with a control, empty expression construct. See FIG. 14. We observed a sharp increase in the amount of mTOR phosphorylated on S2481 in A549 cells expressing LKB1 over the amount seen in control cells.

We will stain the tissue samples described in Additional Lung Cancer Study 1 with an antibody that specifically recognizes LKB1. These antibodies are commercially available from CST and have been validated in IHC applications. We will score the presence of LKB1 with the amount of phospho-S2481 detected in the samples.

We will concurrently analyze several different cancer cell lines that are derived from tumors that are LKB1 deficient for S2481 phosphorylation. We have acquired NCI-H23 cells, which were derived from a non-small cell lung adenocarcinoma, and NCI-H460 cells, which were derived from a large cell lung carcinoma. Like the A549 cell line, these cells are LKB1 null. We will utilize retroviral vector infection to re-introduce WT LKB1 or a kinase-dead (1(D) mutant of LKB1 into these cells. Whole cell lysates will be analyzed by immunoblotting with an antibody specific for mTOR phosphorylated on S2481. Cells reconstituted with an empty expression plasmid will be analyzed in parallel.

c) Additional Lung Cancer Study 3

We will utilize short hairpin RNA (shRNA) to knockdown the expression of specific components of the signaling pathway in the lung tumor cell lines described in Additional Lung Cancer Study 1 and we will analyze the effects on mTORC2 activation. Lentiviral infection will be used to knock down either Raptor, IRS-1 or PI-3 kinase in control cells and cells that have been reconstituted with WT LKB1 (Additional Lung Cancer Study 1). Whole cell lysates will be analyzed by immunoblotting with antibodies that recognize mTOR phosphorylated on S2481. Immunoblots for phospho-S6K and phospho-Akt downstream of mTORC1 and mTORC2, respectively, will be included as controls.

Finally, we will utilize lentiviral shRNA to knockdown the expression of PI-3 kinase to determine functionality in LKB1 signaling to mTORC2. PI-3 kinase is a multi-subunit enzyme with each subunit having several isoforms. Two of the four isoforms of the catalytic subunit, p110α and p110β, are ubiquitously expressed and are involved in insulin receptor signaling. We will begin by depleting p110α and p110β both separately and together, in control and WT LKB1 reconstituted cells, and we will analyze whole cell lysates for the presence of mTOR phosphorylated on S2481.

d) Additional Lung Cancer Study 4

The phosphorylation of mTOR at Ser2481 in lungs/lung tumors from mice that have been exposed to various agents that control mTOR activity will be analyzed. The mouse strains used will be the K-Ras Lox-Stop-Lox (LSL), K-Ras LSL LKB1+/− and K-Ras LSL LKB1−/− models of inducible-lung cancer utilized in Reuben Shaw's laboratory at the Salk Institute (Ji, H. et al. (2007) Nature 448, 807-810). A wild-type mouse strain with appropriate genetic background will be used as a control. Each group of mice will consist of 5 mice aged 6-8 weeks. Mice will be either left untreated, or infected with adenovirus expressing the Cre recombinase (Adeno-Cre) intranasally to induce the expression of the gain-in-function conditional K-Ras allele in lung which will initiate tumor growth (DuPage, M. et al. (2009) Nat Protoc 4, 1064-1072).

Mice will be euthanized at 6, 12 and 16 weeks post-infection. Control mice (no inhalation of Adeno-Cre) will also be analyzed. Mice will be treated with insulin, rapamycin or PP242 by intraperitoneal (IP) injection at time points ranging from 30 minutes to 48 hours prior to euthanasia. Insulin will be administered at a dose of 0.5 units (in a sterile 0.9% saline solution) per kilogram of body weight. Rapamycin (an allosteric inhibitor of mTOR which is expected to decrease S2481 phosphorylation) will be administered at a dose of 5 mg per kilogram of body weight and will be prepared in 100 ml of vehicle containing 20% DMSO, 40% PEG-400 and 40% saline. PP242 (an ATP analog which acts as a competitive inhibitor of mTOR activity) will be administered at a dose of 20 mg per kilogram of body weight and will be prepared in 100 ml of vehicle (Feldman, M. E., et al. PLoS Biol 7, e38). Vehicle alone will be used as a control. Mouse lung tissue will be harvested immediately after euthanasia and will be processed for immunohistochemical analysis of S2481 phosphorylation.

What is claimed is:

1. A method of predicting whether a subject that has a cancer would be responsive to an mTOR inhibition cancer treatment, said method comprising:
(i) detecting a level of phosphorylation of mTOR at serine 2481 in said subject;
(ii) comparing the level of phosphorylation of mTOR at serine 2481 in said subject with a standard control, wherein a high level of phosphorylation of mTOR at serine 2481 in said subject relative to said standard control indicates said subject would be responsive to an mTOR inhibition cancer treatment.

2. A method of monitoring progression of a cancer in a subject that has said cancer, said method comprising:
(i) detecting a level of phosphorylation of mTOR at serine 2481 in said subject;
(ii) comparing the level of phosphorylation of mTOR at serine 2481 in said subject with a standard control, wherein a high level of phosphorylation of mTOR at serine 2481 in said subject relative to said standard control indicates a higher progression of cancer in said subject.

3. A method of determining whether a subject is at risk of developing a cancer, said method comprising:
(i) detecting a level of phosphorylation of mTOR at serine 2481 in said subject;
(ii) comparing the level of phosphorylation of mTOR at serine 2481 in said subject with a standard control, wherein a high level of phosphorylation of mTOR at serine 2481 in said subject relative to said standard control indicates said subject is at risk of developing said cancer.

4. A method of determining whether a subject has a cancer, said method comprising:
(i) detecting a level of phosphorylation of mTOR at serine 2481 in said subject;
(ii) comparing the level of phosphorylation of mTOR at serine 2481 in said subject with a standard control, wherein a high level of phosphorylation of mTOR at serine 2481 in said subject relative to said standard control indicates said subject has said cancer.

5. The method of one of claim 1, 2, 3 or 4, wherein said detecting the level of phosphorylation of mTOR at serine 2481 in said subject comprises detecting a level of phosphorylation of mTOR at serine 2481 in a sample from said subject.

6. The method of claim 5, wherein said detecting the level of phosphorylation of mTOR at serine 2481 in said subject comprises contacting said sample with an anti-S2481 antibody.

7. The method of one of claim 1, 2, 3 or 4, wherein said cancer is breast cancer or lung cancer.

8. The method of one of claim 1, 2, 3 or 4, wherein said standard control is a level of phosphorylation of mTOR at serine 2481 in said subject at an earlier time point.

9. The method of one of claim 1, 2, 3 or 4, wherein said standard control is an average level of phosphorylation of mTOR at serine 2481 derived from a plurality of control subjects.

10. The method of claim 1, wherein said mTOR inhibition cancer treatment is a treatment with rapamycin, Ku-0063794, PP242, PP30, Torin1 or analogs thereof.

11. A method of determining whether a test compound is a cancer therapeutic, said method comprising:
(i) contacting said test compound with a cell;
(ii) detecting a level of phosphorylation of mTOR at serine 2481 in said cell;
(iii) comparing said level of phosphorylation of mTOR at serine 2481 to a standard control, wherein a high level of phosphorylation of mTOR at serine 2481 in said cell relative to said standard control indicates said test compound is a cancer therapeutic.

12. The method of claim 11, wherein said standard control is a level of phosphorylation of mTOR at serine 2481 in said cell in the absence of said test compound.


SEQUENCE LISTING (14 sequences)

SEQ ID NO: 1 - protein, 74 aa, Artificial
  Synthetic peptide of portion of human mTOR near the C-terminus for multiple sequence alignment
PLLNWRLMDTNTKGNKRSRTRTDSYSAGQSVEILDGVELGEPAHKKTGTTVPESIHSFIGDGLVKPEALNKKAI

SEQ ID NO: 2 - protein, 74 aa, Artificial
  Synthetic peptide of portion of oppossum mTOR near the C-terminus for multiple sequence alignment
PLLNWRLMDTNTKGTKRSRTRTDSYSAGQSVELLDSVDLGETAHKKTGTTVPESIHSFIGDGLVKPEALNKKAI

SEQ ID NO: 3 - protein, 73 aa, Artificial
  Synthetic peptide of portion of chicken mTOR near the C-terminus for multiple sequence alignment
PLLNWRLMDTNTKGNKRSRTRTDSYSASQSVEMLDGMELGETAHKKTGTTVPESIHSFIGDGLVKPELNKKAI

SEQ ID NO: 4 - protein, 74 aa, Artificial
  Synthetic peptide of portion of frog mTOR near the C-terminus for multiple sequence alignment
PLLNWRLMDTNTKGNKRSRTRTDSYSAGQSGEIMETVELGETAHKKTGSTVPESIHTFIGDGLVQPEALNKKAI

SEQ ID NO: 5 - protein, 74 aa, Artificial
  Synthetic peptide of portion of zebrafish mTOR near the C-terminus for multiple sequence alignment
PLLNWRLMDTNTKGNKRSRTRTDSYTAGQSVEAMEGIDLGETTHKKPGTTVPESIHSFIGDGLVQPEALNKKAI

SEQ ID NO: 6 - protein, 53 aa, Artificial
  Synthetic peptide of portion of Ciona intestinalis mTOR near the C-terminus for multiple sequence alignment
PLLNWRLVESSEHGRIMESGSISDALGVGHTGIAQTPGETGTARPEVLNKKAL

SEQ ID NO: 7 - protein, 53 aa, Artificial
  Synthetic peptide of portion of Ciona savignyi mTOR near the C-terminus for multiple sequence alignment
PLLNWRLVDNSDHNRVLEGGSVSDALGVGHHNISLAAGETVVARPEVLNKKAL

SEQ ID NO: 8 - protein, 74 aa, Artificial
  Synthetic peptide of portion of Drosophila melanogaster mTOR near the C-terminus for multiple sequence alignment
PLLNWRLLDVDKKGNDAVAGAGAPGGRGGSGMQDSLSNSVEDSLPMAKSKPYDPTLQQGGLHNNVADETNSKAS

SEQ ID NO: 9 - protein, 72 aa, Artificial
  Synthetic peptide of portion of Drosophila virilis mTOR near the C-terminus for multiple sequence alignment
PLLNWRLLDVDKKANDTTATTAAGASGGSPSNSIEQESLLQGNPLAKSKTYEAQQLHVGYTNVADVTNSKAS

SEQ ID NO: 10 - protein, 39 aa, Artificial
  Synthetic peptide of portion of Caenorhabditis elegans mTOR near the C-terminus for multiple sequence alignment
PVINWRLVEGMKKDPKTRKDTGGRQNMAGAVLPSSSTTD

SEQ ID NO: 11 - protein, 43 aa, Artificial
  Synthetic peptide of portion of Caenorhabditis briggsae mTOR near the C-terminus for multiple sequence alignment
PVINWRLVEGMKKDPKAKKEPGGGRAHVIATVNAVLPSTSTTD

SEQ ID NO: 12 - protein, 58 aa, Artificial
  Synthetic peptide of portion of Saccharomyces cerevisiae mTOR1 near the C-terminus for multiple sequence alignment
PLIHWGFDLPPQKLTEQTGIPLPLINPSELLRKGAITVEEAANMEAEQQNETKNARAM

SEQ ID NO: 13 - protein, 58 aa, Artificial
  Synthetic peptide of portion of Saccharomyces cerevisiae mTOR2 near the C-terminus for multiple sequence alignment
PLINWGPDLPTKKIEEETGIQLPVMNANELLSNGAITEEEVQRVENEHKNAIRNARAM

SEQ ID NO: 14 - protein, 2549 aa, Homo sapiens
MLGTGPAAATTAATTSSNVSVLQQFASGLKSRNEETRAKAAKELQHYVTMELREMSQEESTRFYDQLNHHIFELVSSSDANERKGGILAIASLIGVEGGNATRIGRFANYLRNLLPSNDPVVMEMASKAIGRLAMAGDTFTAEYVEFEVKRALEWLGADRNEGRRHAAVLVLRELAISVPTFFFQQVQPFFDNIFVAVWDPKQAIREGAVAALRACLILTTQREPKEMQKPQWYRHTFEEAEKGFDETLAKEKGMNRDDRIHGALLILNELVRISSMEGERLREEMEEITQQQLVHDKYCKDLMGFGTKPRHITPFTSFQAVQPQQSNALVGLLGYSSHQGLMGFGTSPSPAKSTLVESRCCRDLMEEKFDQVCQWVLKCRNSKNSLIQMTILNLLPRLAAFRPSAFTDTQYLQDTMNHVLSCVKKEKERTAAFQALGLLSVAVRSEFKVYLPRVLDIIRAALPPKDFAHKRQKAMQVDATVFTCISMLARAMGPGIQQDIKELLEPMLAVGLSPALTAVLYDLSRQIPQLKKDIQDGLLKMLSLVLMHKPLRHPGMPKGLAHQLASPGLTTLPEASDVGSITLALRTLGSFEFEGHSLTQFVRHCADHFLNSEHKEIRMEAARTCSRLLTPSIHLISGHAHVVSQTAVQVVADVLSKLLVVGITDPDPDIRYCVLASLDERFDAHLAQAENLQALFVALNDQVFEIRELAICTVGRLSSMNPAFVMPFLRKMLIQILTELEHSGIGRIKEQSARMLGHLVSNAPRLIRPYMEPILKALILKLKDPDPDPNPGVINNVLATIGELAQVSGLEMRKWVDELFIIIMDMLQDSSLLAKRQVALWTLGQLVASTGYVVEPYRKYPTLLEVLLNFLKTEQNQGTRREAIRVLGLLGALDPYKHKVNIGMIDQSRDASAVSLSESKSSQDSSDYSTSEMLVNMGNLPLDEFYPAVSMVALMRIFRDQSLSHHHTMVVQAITFIFKSLGLKCVQFLPQVMPTFLNVIRVCDGAIREFLFQQLGMLVSFVKSHIRPYMDEIVTLMREFWVMNTSIQSTIILLIEQIVVALGGEFKLYLPQLIPHMLRVFMHDNSPGRIVSIKLLAAIQLFGANLDDYLHLLLPPIVKLFDAPEAPLPSRKAALETVDRLTESLDFTDYASRIIHPIVRTLDQSPELRSTAMDTLSSLVFQLGKKYQIFIPMVNKVLVRHRINHQRYDVLICRIVKGYTLADEEEDPLIYQHRMLRSGQGDALASGPVETGPMKKLHVSTINLQKAWGAARRVSKDDWLEWLRRLSLELLKDSSSPSLRSCWALAQAYNPMARDLFNAAFVSCWSELNEDQQDELIRSIELALTSQDIAEVTQTLLNLAEFMEHSDKGPLPLRDDNGIVLLGERAAKCRAYAKALHYKELEFQKGPTPAILESLISINNKLQQPEAAAGVLEYAMKHFGELEIQATWYEKLHEWEDALVAYDKKMDTNKDDPELMLGRMRCLEALGEWGQLHQQCCEKWTLVNDETQAKMARMAAAAAWGLGQWDSMEEYTCMIPRDTHDGAFYRAVLALHQDLFSLAQQCIDKARDLLDAELTAMAGESYSRAYGAMVSCHMLSELEEVIQYKLVPERREIIRQIWWERLQGCQRIVEDWQKILMVRSLVVSPHEDMRTWLKYASLCGKSGRLALAHKTLVLLLGVDPSRQLDHPLPTVHPQVTYAYMKNMWKSARKIDAFQHMQHFVQTMQQQAQHAIATEDQQHKQELHKLMARCFLKLGEWQLNLQGINESTIPKVLQYYSAATEHDRSWYKAWHAWAVMNFEAVLHYKHQNQARDEKKKLRHASGANITNATTAATTAATATTTASTEGSNSESEAESTENSPTPSPLQKKVTEDLSKTLLMYTVPAVQGFFRSISLSRGNNLQDTLRVLTLWFDYGHWPDVNEALVEGVKAIQIDTWLQVIPQLIARIDTPRPLVGRLIHQLLTDIGRYHPQALIYPLTVASKSTTTARHNAANKILKNMCEHSNTLVQQAMMVSEELIRVAILWHEMWHEGLEEASRLYFGERNVKGMFEVLEPLHAMMERGPQTLKETSFNQAYGRDLMEAQEWCRKYMKSGNVKDLTQAWDLYYHVFRRISKQLPQLTSLELQYVSPKLLMCRDLELAVPGTYDPNQPIIRIQSIAPSLQVITSKQRPRKLTLMGSNGHEFVFLLKGHEDLRQDERVMQLFGLVNTLLANDPTSLRKNLSIQRYAVIPLSTNSGLIGWVPHCDTLHALIRDYREKKKILLNIEHRIMLRMAPDYDHLTLMQKVEVFEHAVNNTAGDDLAKLLWLKSPSSEVWFDRRTNYTRSLAVMSMVGYILGLGDRHPSNLMLDRLSGKILHIDFGDCFEVAMTREKFPEKIPFRLTRMLTNAMEVTGLDGNYRITCHTVMEVLREHKDSVMAVLEAFVYDPLLNWRLMDTNTKGNKRSRTRTDSYSAGQSVEILDGVELGEPAHKKTGTTVPESIHSFIGDGLVKPEALNKKAIQIINRVRDKLTGRDFSHDDTLDVPTQVELLIKQATSHENLCQCYIGWCPFW